(19) United States
(12) Patent Application Publication
(10) Pub. No.: US 2009/0209937 A1
(43) Pub. Date: Aug. 20, 2009

(54) Apparatus and Methods for Treating Epilepsy Using Convection-Enhanced Delivery

(75) Inventors: Michael Andrew ROGAWSKI, Sacramento, CA (US); Maciej GASIOR, Devon, PA (US)

Correspondence Address: King Spalding LLP, 4 Embarcadero Center, Suite 3500, San Francisco, CA, 94111, US

(73) Assignee: ARGENIS LLC, Sacramento, CA (US)

(21) Appl. No.: 12/189,063

(22) Filed: Aug. 8, 2008

Related U.S. Application Data
(60) Provisional application No. 60/955,361, filed on Aug. 11, 2007.

(51) Int. Cl. A61M 5/168; A61K 38/17; A61K 39/08; A61K 31/421; A61K 9/127; A61M 5/14

(52) U.S. Cl. 604/503; 514/12; 424/239.1; 514/376; 424/450; 604/506

(57) ABSTRACT

Disclosed herein are apparatuses and methods for treating a neurological disorder associated with excessive neuronal excitability (e.g., epilepsy). Methods disclosed herein comprise administering to a subject in need of such treatment an antiepileptic drug solution comprising a therapeutically effective amount of an antiepileptic drug (e.g., a toxin that inhibits the exocytosis of neurotransmifters, excitotoxins, etc.) using convection-enhanced delivery (CED).

### RELATED APPLICATIONS

This application claims the benefit under 35 U.S.C. § 119(e) to U.S. Provisional Patent Application No. 60/955,361, filed Aug. 11, 2007, and entitled “Convection-enhanced Delivery of Anticonvulsant Toxins to Treat Partial Epilepsy,” which is incorporated herein by reference in its entirety.

### GOVERNMENT INTEREST

The invention was developed with U.S. Government support from the National Institutes of Health. Thus, the U.S. Government may have certain rights in the invention.

### TECHNICAL FIELD

Described herein are apparatuses and methods for treating neurological disorders associated with excessive neuronal excitability (e.g., epilepsy) by administration of antiepileptic drugs via convection-enhanced delivery (CED).

### BACKGROUND

Mesial temporal lobe epilepsy (TLE), the most common type of focal epilepsy, remains a significant therapeutic challenge (Nadkarni, S., et al. (2005) Neurology 64:S2-11). It is estimated that 30% of TLE patients are resistant to currently available antiepileptic drugs (Berg, A. T., et al. (2003) Epilepsia 44:1425-33). Although resective surgery can lead to seizure control in some of these refractory patients, not all such individuals are candidates for surgery, and even among those considered to be candidates, a substantial portion (17%) will refuse to accept the risks of a major surgical procedure (Id.). The ultimate limiting factor in conventional systemic antiepileptic drug therapy is the unwanted side effects of the therapeutic agent on brain systems not involved in the epileptic process. In addition, systemic toxicities and teratogenicity may also be a concern.

In focal epilepsies like TLE, it may be possible to minimize or avoid side effects and systemic toxicities by restricting delivery of the therapeutic agent to a limited region of brain (Nilsen, K. E. and Cock, H. R. (2004) Brain Res. Rev. 44:141-153; see also U.S. Pat. No. 7,357,934). The success of resective surgery indicates that local targeting has the potential to provide therapeutic benefit. However, bolus injection of solutions into the brain parenchyma as proposed in the '934 patent can cause local tissue deformation and damage, provides poor control over the extent of tissue exposure, and the resulting drug concentrations in the exposed region are highly inhomogeneous. Further, injection of aqueous solutions or implantation of controlled release formulations provides little to no control over the diffusion of the therapeutic agent, which may be particularly important if the therapeutic agent is highly toxic, and moreover the duration of action with these local therapies is insufficient.

In brain slice preparations, N-type calcium channel antagonists, e.g., conotoxins, inhibit epileptiform activity by suppressing synaptic transmission (Boulton, C. L. and O'Shaughnessy, C. T. (1991) Eur J. Neurosci. 3:992-1000; Kamiya, H., et al. (1988) Neurosci. Lett. 91:84-88; Dutar, P. et al., (1989) Eur. J. Pharmacol. 174:261-266; Wu, L. G. and Saggau, P. (1994) J. Neurosci. 14:5613-22). However, the toxins have limited bioavailability and distribute poorly in the brain so that they are not active systemically (Olivera B. M., et al. (1985) Science 230:1338-43; Miljanich, G. P., and Ramachandran, J. (1995) Annu. Rev. Pharmacol. Toxicol. 35:707-34; Newcomb, R., (2000) Peptides 21:491-501). Although they do have anticonvulsant activity when administered at high doses intraventricularly, these clinically effective doses are also invariably associated with profound generalized tremor (Jackson, H. C. and Scheideler, M. A. (1996) Psychopharmacology (Berl) 126:85-90).

What is needed, therefore, is an alternative modality for delivering antiepileptic drugs to the brain such that the clinically desirable anticonvulsant effect can be maximized, but without the attendant toxicities found with the current treatment modalities employed in the art, and with a sufficiently long duration of action to be clinically practical.

### SUMMARY

Disclosed herein is the applicability of convection-enhanced delivery (CED) for the treatment of focal epilepsy through the local delivery of antiepileptic drugs, (e.g., anticonvulsant toxins, excitotoxins, and the like). As demonstrated herein for the first time, Applicants surprisingly discovered that CED administration of even severely toxic antiepileptic drugs can be used to effectively treat epilepsy such that little or no toxicity is observed, and further that the effect can be maintained for an extended period of time, e.g., up to several months, thereby making clinical treatment of epilepsy more feasible.

Accordingly, described herein are apparatuses and methods of treating a neurological disorder associated with excessive neuronal excitability comprising administering to a subject in need of such treatment a therapeutically effective dose of an antiepileptic drug using CED. In one embodiment, the neurological disorder is a type of epilepsy, e.g., a type of epilepsy selected from the group consisting of partial epilepsy, simple partial seizures, Jacksonian seizures, and complex partial (psychomotor) seizures.

Any suitable antiepileptic drugs known in the art may be used in the methods and apparatuses of the present invention, as described herein. Preferred antiepileptic drug are those having anticonvulsant properties and/or that diminish or inhibit epileptiform discharges.

In one embodiment, the antiepileptic drug is selected from the group consisting of an anticonvulsant toxin and an excitotoxin. Suitable excitotoxins include, e.g., ibotenate, while suitable anticonvulsant toxins include those that inhibit exocytosis of neurotransmitters such as, e.g., conotoxins, botulinum toxins, conantokins, etc., as well as derivatives and pharmaceutically acceptable salts thereof. Preferred anticonvulsant toxins are those that do not destroy the cell.

In one embodiment, the anticonvulsant toxin is an ω-conotoxin, e.g. ω-conotoxin MVIIA or ω-conotoxin GVIA. In another embodiment, the anticonvulsant toxin is a botulinum toxin, e.g., a botulinum toxin serotype A such as Botox® or Dysport®, a botulinum toxin serotype B such as Myobloc®, etc. In another embodiment, the anticonvulsant toxin is a μ-conotoxin or a conantokin peptide.

In an alternative embodiment, the antiepileptic drug is an excitotoxin, e.g., ibotenate, kainate, quinolinate, and the like.

A single antiepileptic drug or appropriate combinations thereof may be administered in any pharmaceutically acceptable formulation including, e.g., liposomal delivery vehicles and the like. Further, the drugs may be administered alone or in conjunction with a tracer molecule, either in the same or a different formulation than that including the antiepileptic drug.

In one embodiment, a pharmaceutically acceptable formulation comprising the antiepileptic drug is administered with a pump and a catheter, wherein the pump is external to the body, wherein the catheter enters the body percutaneously, and wherein a proximal end of the catheter is coupled to the pump. In another embodiment, the antiepileptic drug formulation may be administered using an apparatus comprising a combination implantable pump and catheter, a control unit, and a sensor that detects electrical interictal or ictal seizure activity and which, through the control unit, commands the pump to deliver the drug formulation when needed. In another embodiment, the control unit may store a database of the sensor output signals and provide a signal to the pump based on the continuously updated database.

In one embodiment, the antiepileptic drug formulation is administered through multiple distal infusion catheters with discharge ports situated in different regions of epileptic focus or in different foci, if more than one foci are in need of treatment. In a further embodiment, multiple indwelling intraparenchymal multiport brain catheters are employed having one or more transdermal ports. See. e.g., U.S. Patent Publication No. 2005/0137134, the disclosure of which is expressly incorporated herein in its entirety.

### BRIEF DESCRIPTION OF THE DRAWINGS

Further objects, features and advantages of the apparatuses and methods described herein will become apparent from the following detailed description taken in conjunction with the accompanying figures showing illustrative embodiments of the invention.

FIG. 1 shows components of a cannula-bipolar stimulating electrode assembly system that may be used for kindling stimulation and recording for convection enhanced delivery (CED) delivery of solutions (e.g., an antiepileptic drug solution) into foci of the brain, e.g., the basolateral amygdala. The combination guide cannula and bipolar stimulating assembly is shown to the right. The guide cannula passes through the threaded left-most pedestal; stimulating electrodes are affixed diametrically opposed to the exterior of the guide cannula below the pedestal. The two wire electrodes pass to the right-most pedestal containing pin connectors to allow electrical connection to the kindling stimulator. The dummy cannula wire-cap assembly is shown to the left. The plastic cap is internally threaded to match the treads of the pedestal of the cannula-electrode assembly. The CED cannula is shown in the center. The plastic stop maintains the tip of the CED cannula 0.5 mm above the tips of the stimulating electrode wires.

FIG. 2 shows mean relative afterdischarge (AD) threshold, AD duration, seizure stage, and duration of behavioral seizures values at time intervals from 20 min to 1 week (x-axes) after CED infusion of ω-conotoxin-GVIA (ω-CTX-G; 0.005, 0.05, 0.5 nmol/infusion) or vehicle. Mean relative AD threshold, AD duration, seizure stage and duration are represented as Percent Change From Baseline (y-axes) and values were calculated as an average of the values obtained in response to at least 2 stimulation sessions prior to the infusion. Each data point represents a mean±S.E.M. of values from 10 rats. *, Significantly different from vehicle value at that time point (Tukey test) following the detection of a statistically significant main effect by a two-way repeated measures ANOVA. Results of ANOVA and post-hoc analysis are presented in Table 1.

FIG. 3 shows the relative AD threshold, AD duration, seizure stage, and duration of behavioral seizures at time intervals from 20 min to 1 week (x-axes) after CED infusion of ω-conotoxin MVIIA (ω-CTX-M; 0.05, 0.15, 0.5 nmol/infusion) or vehicle. Mean relative AD threshold, AD duration, seizure stage and duration are represented as are represented as a Percent Change From Baseline (y-axes) and values are calculated as an average of the values obtained in response to at least 2 stimulation sessions prior to the infusion. Each data point represents a mean±S.E.M. of values from 10-12 rats. *, Significantly different from vehicle value at that time point (Tukey test) following the detection of a statistically significant main effect by a two-way repeated measures ANOVA. Results of ANOVA and post-hoc analysis are presented in Table 1.

FIG. 4 shows Area-under-the-curve (AUC; y-axes) analysis of the data from FIGS. 2 and 3. The area-under-the-curve of the percent change values for the period 20 min to 1 week (x-axes) following infusion for every time-course data set in each animal were determined using the trapezoidal method. The data points represent the mean±S.E.M. of the values (in percent-days) for all animals receiving a specific toxin and dose. Open squares (□) and circles (◯) indicate control values for the group of animals tested with ω-CTX-GVIA (ω-CTX-G; ▪) and ω-CTX-MVIIA (ω-CTX-M; ), respectively. *, Significantly different from corresponding vehicle value at that dose (Tukey test) following the detection of a statistically significant main effect by a two-way repeated measures ANOVA.

FIG. 5. shows baseline values for Current Intensity (μA; y-axis), duration (sec; y-axis), stage (y-axis), and duration (sec; y-axis) respectively for AD threshold, AD duration, seizure stage, and duration of behavioral seizures before each of four sequential CED infusions (test sessions I-IV; x-axes) of ω-CTX-GVIA and ω-CTX-MVIIA at various doses and vehicle in the experiments presented in FIGS. 2 and 3. The values for test session I represent the kindling measures for 22 animals prior to any infusion (true baseline). The treatments in later test session (II-IV) were presented in random order. The baseline values were calculated by averaging seizure measures in response to at least 3 electrical stimulations prior to an infusion. Each bar represents the mean±S.E.M. There were no significant differences between the mean values of any of the seizure measures among the four test sessions (two-way repeated measures ANOVA), indicating that the baseline prior to CED infusion was stable during repeated CED infusion and testing.

FIG. 6 shows cresyl violet stained coronal brain sections (top images) and adjacent silver stained brain sections (bottom images) of (A) brain obtained from an animal that had been fully kindled, had received three CED infusions of ω-CTX-G and an additional vehicle infusion CED or (B) brain obtained from a rat infused with 0.05 nmol ω-CTX-G at a rate of 2.5 μL/min (10-fold greater than for CED). The images suggest that CED causes minimal damage in the amygdala and surrounding brain structures in contrast to bolus infusion which causes cavitation. A, Brain obtained from an animal that had been fully kindled, had received three CED infusions of ω-CTX-G and an additional vehicle infusion. The animal had experienced multiple stimulation-induced kindled seizures. Top, cresyl violet stained coronal section through the track of the cannula-electrode assembly and right amygdala. Arrow indicates location of track. There is minimal tissue disruption along the track and no damage apparent in the amygdala. Bottom, the adjacent silver stained section shows some staining in the right striatum, thalamus, and internal capsule, indicating neuronal disintegration. There is no damage apparent in the amygdala. B, Brain obtained from a rat infused with 0.05 nmol ω-CTX-G at a rate of 2.5 μL/min (10-fold greater than for CED). Tracks of the stimulating electrode wires are seen to extend into the right basolateral amygdala and a cavity is present above the nucleus. A second cavity is present in the alveus due to damage from the guide cannula and tracking of the infused solution up the cannula. Cavitation was observed in 7 of 8 rats treated in a similar fashion.

FIG. 7 represents an assessment of the influence of repeated kindling stimulation on recovery from the toxin effects. Shown are the Current Intensity (μA; y-axis), Duration in seconds (sec; y-axis), Stage (y-axis) and Duration in seconds (sec; y-axis) respectively of AD threshold, AD duration, seizure stage, and duration of behavioral seizures before the infusion (baseline control values) and 7 and 8 days after CED infusion (x-axis) of vehicle, ω-CTX-G (0.05 nmol) and ω-CTX-M (0.5 nmol). After the infusions, the animals were not electrically stimulated until 7 days after the infusion. Bars represent the mean±S.E.M. of the values from 6 rats. Baseline values for each animal are averages from at least 3 electrical stimulations prior to the infusion.

FIG. 8 shows the mean relative (Percent Change From Baseline; y-axis) AD threshold, AD duration, seizure stage, and duration of behavioral seizures values at intervals from 20 min to 72 h (x-axis) after CED infusion of proteolyzed ω-CTX-M (8 rats) or vehicle (8 rats). Each data point represents a mean±S.E.M. There were no significant differences between the values for the proteolyzed toxin and vehicle by a two-way repeated measures ANOVA. The data indicate a lack of effect of CED infusion of proteolyzed ω-CTX-M (0.5 nmol) on kindling measures.

FIG. 9. shows the mean relative (Percent Change From Baseline; y-axes) AD threshold, AD duration, seizure stage, and duration of behavioral seizures values at intervals from 20 min to 1 week α-axes) after CED infusion of 500 nmol carbamazepine (8 rats) or vehicle (6 rats). *, Significantly different from vehicle value (Tukey test) following the detection of a statistically significant main effect by a two-way repeated measures ANOVA.

FIG. 10 shows the effects of CED infusion of ω-CTX-G (0.05 nmol), ω-CTX-M (0.5 nmol) or vehicle on locomotor activity at intervals from 20 min to 96 h (x-axis) after the infusion presented as the percent change from baseline (right panels). Each data point represents the mean±S.E.M. of experiments with 6 rats. The dotted lines represent no change from the baseline values. The 18 animals were habituated to the locomotor-activity chamber in 5 daily sessions prior to the infusion (left panels). Baseline values were the averages of the activity counts during the last 2 habituation sessions.

FIG. 11 shows the afterdischarge threshold (AD threshold) and afterdischarge duration (AD duration) values (in μA and sec, respectively) 2-4 days before CED (basline values) and 3 to 64 days after CED infusion of botulinum neurotoxins A (left plots) and B (right plots) or vehicle. Each toxin was administered once in the dose of 1, 3.2, or 10 ng. Each dose was administered in a separate group of fully kindled rats (n=7 per each dose); vehicle was administered in control rats (n=8). Each data point represents a mean of values from 7 to 8 rats. *, Significantly different from vehicle value at that time point (Tukey test) following the detection of a statistically significant main effect by a two-way repeated measures ANOVA. Results of ANOVA and post-hoc analysis are presented in Table 2.

FIG. 12 shows area-under-the-curve (AUC) analysis of the data for changes in AD threshold (upper left plot), AD duration (upper right plot), seizure stage (lower left plot), and duration of behavioral seizures (lower right plot) from FIG. 11. The area-under-the-curve was calculated for each dose and rat separately based on the percent change values for the period of 3 to 64 days following CED infusion relative to rat's individual baseline values prior to CED. The graphs plot the mean±S.E.M. of these values for all animals receiving a specific toxin and dose. Solid circle () and square (▪) indicate BTX A and BTX B, respectively (1-10 ng); open square (□) represents vehicle-treated control group. *, Significantly different from vehicle value (Dunnett's test) following the detection of a statistically significant main effect by a one-way ANOVA.

FIG. 13 is a schematic representation of one embodiment described herein.

### DETAILED DESCRIPTION

Described herein are apparatuses and methods of using antiepileptic drugs, including but not limited to anticonvulsant toxins (e.g., conotoxins, botulinum toxins, conantokins, etc.) and excitotoxins (e.g., ibotenate) to treat conditions such as epilepsy or other neurological disorders associated with excessive neuronal excitability. Such methods of treatment comprise administering to a subject in need of such treatment antiepileptic drugs (e.g., in pharmaceutically acceptable formulations) using convection-enhanced delivery (CED).

The term “epilepsy” as used herein refers to a disorder of brain function characterized by the occurrence of seizures. The term “seizure” as used herein refers to a transient alteration of behavior due to the synchronous and rhythmic firing of populations of brain neurons. Types of epilepsy that can be treated or prevented include, but are not limited to, partial epilepsy, including but not limited to, simple partial seizures, Jacksonian seizures, and complex partial (psychomotor) seizures. The method also is useful to treat or prevent secondary generalized convulsive seizures (grand mal or tonic-clonic seizures).

The methods described herein are also effective in the treatment of disorders of the central nervous system other than epilepsy, including migraine, chronic pain, neuropathic pain, myoclonus, essential tremor, dyskinesia, including but not limited to tardive dyskinesia, and other movement disorders, Parkinson's disease (including the symptoms associated with Parkinson's disease, including but not limited to, bradykinesia, muscular rigidity, resting tremor, and impairment of postural balance) and muscle spasm.

### Administration of Antiepileptic Drugs Via CED

The antiepileptic drugs effective in this method often cause neurological impairment and other undesirable neurological side effects (e.g., tremor) when administered into the cerebrospinal fluid. Anticonvulsant toxins in particular may also cause localized brain damage when administered by bolus injection or similar diffusion-based delivery modalities as described in the prior art, such as the direct intraparenchymal injection or polymer implants described in U.S. Pat. No. 7,357,934. The toxins are either ineffective or are associated with unacceptable side effects when administered by techniques such as these that depend on diffusion for the toxin molecules to reach the target sites, because such techniques typically produce a concentration gradient with high toxin concentrations at the site of delivery and progressively lower concentrations at distal sites within the epileptic focus, that is not suitable for seizure protection without undesirable neurological side effects.

Applicants have discovered that the antiepileptic drugs described herein, e.g., anticonvulsant toxins, excitotoxins, and the like may protect against partial seizures when administered into the seizure focus using CED. Importantly, the antiepileptic drugs may be administered by local administration, that is directly to the site where a therapeutic effect is desired. CED provides a more uniform distribution of antiepileptic drugs than does diffusion, and the distribution may be better controlled to the region required for the desired therapeutic effect. Applicants have unexpectedly found that such a uniform distribution, as provided by CED, is required to obtain adequate seizure protection. Applicants have also unexpectedly found that at appropriate doses and when delivered using CED, the antiepileptic drugs described herein protect against seizures without observable neurological toxicity even though the antiepileptic drugs are toxic themselves. The present invention may thus be useful for a variety of antiepileptic drugs or combinations thereof that suppresses or inhibit epileptic activity of the brain (including seizure discharges and interictal epileptiform discharges).

Applicants believe that they are the first to show that certain antiepileptic drugs described herein can have long-lasting anticonvulsant effects when administered using CED. For example, more conventional antiepileptic drugs such as carbamazepine when delivered by CED may be protective against seizures for only a few hours, as the drug diffuses rapidly away from the site of delivery. In contrast, the prolonged duration of action achieved by convection enhanced delivery of the anticonvulsant toxins described herein provides a more clinically practicable approach, since a single infusion can provide many months of seizure protection.

Significantly, as also demonstrated herein for the first time, Applicants have discovered that anticonvulsant toxins which ordinarily produce severe neurological toxicity when injected into the CSF (such as intense tremor in the case of the ω-conotoxins) are completely free of untoward neurological side effects when their delivery is restricted to a limited region of brain parenchyma by the use of a controlled CED infusion. Accordingly, the experimental results provided herein demonstrate that it is possible to obtain strong and persistent effects on seizure threshold without any observable neurological or behavioral side effects

Accordingly, described herein are apparatuses and methods for administering antiepileptic drugs into the brain to treat neurological disorders, e.g., epilepsy, via CED.

### Convection-Enhanced Delivery

Convection-enhanced delivery (CED) is a direct intracranial drug delivery technique that utilizes a bulk-flow mechanism to deliver and distribute macromolecules to clinically significant volumes of solid tissues. CED offers a greater volume of distribution than simple diffusion and is designed to direct a drug to a specific target site. See, e.g., U.S. Pat. No. 5,720,720, the disclosure of which is expressly incorporated by reference herein. Briefly, convection-enhanced delivery (CED) is a method that circumvents the blood-brain barrier and allows large molecular weight substances to be administered uniformly and in a controlled fashion within a defined region of brain. CED may be used to administer a fluid pharmacological agent (e.g., antiepileptic drug formulation) to a solid tissue (e.g., the brain) through direct convective interstitial infusion and over a predetermined time by inserting a catheter directly into the tissue; and administering the agent under pressure through the catheter into the interstitial space at a predetermined flow rate, e.g., from about 0.5 μL/min to about 15 μL/min.

As detailed herein, Applicants have discovered that CED may be used for treating pharmacoresistant (i.e., intractable) partial epilepsy (i.e., epilepsy where a brain focus can be identified), particularly as a replacement for resective surgery, such as temporal lobectomy or resection of an extratemporal focus. As described in the Examples section, CED of antiepileptic drugs over a short period of time (e.g., 20 min) produces a protective effect that may persist for several days to nearly two months.

A suitable apparatus that may be used for administration of an antiepileptic drug may comprise a pump device that contains a reservoir filled with an antiepileptic drug formulation comprising the antiepileptic drug. The pump may be external to the body or implanted within the body. The pump may be connected to a catheter, which may be implanted into an epileptic focus within the brain. The pump may be activated to release the antiepileptic drug formulation at a pressure and flow rate that causes the solute to convect within the focus, leading to seizure protection. Depending upon the type of antiepileptic drug delivered, the seizure protection may last for several days to many months (typically two to as many as six months).

The duration and other parameters of the infusion may be adjusted to distribute the antiepileptic drug throughout the epileptic focus but not distribute to brain nuclei beyond the epileptic focus, and not into the cerebrospinal fluid. Depending upon the size and shape of the epileptic focus, it may be necessary to use multiple implanted infusion catheters or to use an infusion catheter with multiple solution exit ports. At the conclusion of the period of efficacy, ordinarily signaled by the return of seizures or electrographic signs [as detected by an electroencephalogram (EEG) recording] of increasing electrical excitability within the epileptic focus, the pump may be reactivated to deliver an additional dose of the antiepileptic drug. This reactivation may be continued as necessary until the fluid within the pump is exhausted. The pump may then be refilled or a new pump implanted.

Using CED, an antiepileptic drug may be distributed by slow infusion of a suitable formulation comprising the antiepileptic drug into the interstitial space under positive pressure through a fine cannula. Bulk flow driven by hydrostatic pressure derived from a pump may be used to distribute the antiepileptic drug within the extracellular spaces of the CNS. Since CED does not rely on diffusion for distribution of the infused agents, it is not limited by the molecular weight, concentration, or diffusivity of the antiepileptic drug. Because the use of CED permits distribution of antiepileptic drugs directly within nervous tissues via the tip of a cannula, the blood-brain barrier is bypassed and specific regions in the central nervous system may be targeted, including different regions defined as epileptic focus or as identified for resection by a conventional presurgical evaluation, and in different foci if more than one foci are in need of treatment. Based on the properties of bulk flow, CED may be used to distribute both small and large antiepileptic drugs reliably, safely, and homogeneously over a range of volumes. Further, unlike the intraparenchymal injection proposed in the prior art, CED does not cause structural or functional damage to the infused tissue and provides greater control over the distribution of the antiepileptic drug. In CED, antiepileptic drugs may be distributed homogeneously throughout a distribution volume that is proportional to the infusion volume regardless of the molecular weight of the antiepileptic drug. CED is particularly applicable to slowly diffusing antiepileptic drugs of high molecular weight since the distribution of such large antiepileptic drugs is effectively restricted to the region of the infusion.

In one embodiment described herein, CED may be applied with a small diameter catheter permanently implanted in the brain region using an infusion pump. Antiepileptic drugs to be administered may be prepared as an aqueous isotonic solution, or other appropriate formulation. As described in the Examples section, Applicants discovered that brief (20 min) CED infusions of toxins (e.g., ω-conotoxin GVIA, ω-conotoxin MVIIA (ziconotide), botulinum neurotoxins A and B, etc.) can elevate the seizure threshold in kindled rats for up to two months without side effects.

In one embodiment, an ultrafine (0.2 mm OD at tip), minimally traumatic catheter system specially designed for transcutaneous CED delivery may be used. The catheter system has a step design, which may eliminate solution reflux along the sides of the catheter. Such solution leakage is a major problem with straight-sided catheters. The catheter system may be constructed of polyurethane and fused silica so that it is highly biocompatible and does not interfere with MRI signals. With a long acting antiepileptic drug such an anticonvulsant toxin, readministration is expected to be necessary at approximately three month intervals. The transcutaneous port remains capped during the interval period and is covered with hair so that it may have negligible cosmetic impact. Multiple catheter designs are also feasible so that a larger area of brain can be perfused than is feasible with a single catheter.

In one embodiment, an infusion volume of about 100 μL provides a distribution volume of 250 mm³ in the primate brain.

In one embodiment, there are multiple distal infusion catheters with discharge ports situated in different regions of the epileptic focus or in different foci, if more than one foci are in need of treatment. In a further embodiment, multiple indwelling intraparenchymal multiport brain catheters are employed having one or more transdermal ports. See. e.g., U.S. Patent Publication No. 2005/0137134.

In one embodiment, the invention employs a pump that is external to the body and a catheter that enters the body percutaneously. The proximal end of the catheter is coupled to the pump.

In one embodiment, the invention employs a combination implantable pump and catheter, a control unit, and a sensor that detects electrical interictal or ictal seizure activity and which, through the control unit, commands the pump to deliver toxin solution when needed. The control unit may store a database of the sensor output signals and it provides a signal to the pump based on the continuously updated database.

Generally, antiepileptic drugs may be administered via CED in a pharmaceutically acceptable formulation (e.g., antiepileptic drug solution) comprising a therapeutically effective amount of the antiepileptic drug. In some embodiments, the antiepileptic drug may be contained within liposomes or another appropriate carrier having properties desirable for CED infusion into the brain. An “antiepileptic drug formulation” as used herein refers to a pharmaceutically acceptable formulation comprising antiepileptic drugs in the broad class of substances that diminish or inhibit epileptiform discharges.

Ideal CED antiepileptic drugs should be very potent because a small volume (with a small amount of solute) is delivered to the target site. Further, a hydrophilic antiepileptic drug may advantageously be used, as the hydrophilicity of the antiepileptic drug may aid in its efficacy in treating neurological disorders associated with excessive neuronal excitability, e.g., by preventing the diffusion of the antiepileptic drug through brain tissue. Therefore, when such antiepileptic drugs are deposited by CED, the antiepileptic drugs may remain at the site of deposition for long periods of time and may contribute to the prolonged duration of action observed in the studies detailed in the Examples section.

### Antiepileptic Drugs

As used herein, the term “antiepileptic drug” refers to agents having anticonvulsant properties or that diminish or inhibit epileptiform discharges. In one embodiment, the antiepileptic drug is an excitotoxin, e.g., ibotenate, kainite, quinolinate, domoate, etc. See, e.g., Pace et al., J. Neurosurg. 2002 August; 97(2):450-4. In one embodiment, the antiepileptic drug is an anticonvulsant toxin, e.g., a neuronal exocytosis-inhibiting toxin, including toxins that inhibit exocytosis by blocking ion channels required for calcium entry into nerve terminals or those that inhibit the vesicular release machinery, including botulinum toxins that target proteins required for vesicular release, including synaptosomal associated protein 25 (SNAP-25), vesicle-associated protein VAMP (synaptobrevin), and syntaxin. Preferred embodiments of anticonvulsant toxins for use in the subject invention will inhibit exocytosis without permanently destroying the cell.

Non-limiting examples of anticonvulsant toxins that may be used to treat neurological disorders associated with excessive neuronal excitability (e.g., epilepsy) include μ-conotoxins (e.g., μ-conotoxin GIIIA, μ-conotoxin GIIIB, μ-conotoxin GIIIC, μ-conotoxin PIIIA, μ-conotoxin SmIIIA, μ-conotoxin KIIIA, etc.), ω-conotoxins (e.g., ω-conotoxin GVIA (also referred to herein as “ω-conotoxin G” and “ω-CTX-G”), ω-conotoxin MVIIA (also referred to herein as “ω-conotoxin M” and “ω-CTX-M”), botulinum toxins (e.g., botulinum toxin A (also referred to herein as BTX-A), botulinum toxin B (also referred to herein as “BTX-B”, botulinum toxin C1, botulinum toxin D, botulinum toxin E, botulinum toxin F, etc.), conantokin peptides (e.g., conantokin G, conantokin T, conantokin L, conantokin S1, conantokin Oc, conantokin Gm, conantokin Ca2, conantokin Ca1, and conantokin Qu), derivatives thereof, and pharmaceutically acceptable salts thereof.

In another embodiment, antiepileptic drugs for use in the subject invention also encompass gene therapy vectors capable of expressing proteins having anticonvulsant properties and/or that diminish or inhibit epileptiform discharges, e.g., neuropeptide Y, galanin, etc. See, e.g., Noèet al. (2008) Brain June; 131(Pt 6):1506-15 (Epub 2008 May 13); Foti et al. (2007) Gene Ther November; 14(21):1534-6; McCown T J (2004) Expert Opin Biol Ther. November; 4(11):1771-6; Haberman et al. (2003) Nat. Med. August; 9(8):1076-80; Richichi et al. (2004) J. Neurosci. March 24; 24(12):3051-9.

Conotoxins

Mollusks of the genus Conus produce a highly toxic venom that enables them to immobilize their prey by injecting them with the venom. These venoms disrupt essential organ systems in the envenomated animal, and many of these venoms contain molecules directed to receptors and ion channels of neuromuscular systems. The cone snails that produce these toxic peptides, which are generally referred to as conotoxins, are a large genus of venomous gastropods comprising approximately 500 species. Several peptides isolated from Conus venoms have been characterized. Each Conus species venom appears to contain a unique set of 50 to 200 peptides. The composition of the venom differs greatly between species and between individual snails within each species, each optimally evolved to paralyze it's prey. The active components of the venom are small peptides toxins, typically 10 to 30 amino acid residues in length and are typically highly constrained peptides due to their high density of disulphide bonds. The venom components act on voltage-gated ion channels, ligand-gated ion channels, and G protein-coupled receptors. The pharmaceutical selectivity of conotoxins is at least in part determined by specific disulfide bond frameworks combined with hypervariable amino acids within disulfide loops. Due to the high potency and exquisite selectivity of the conotoxin peptides, several have been evaluated for the treatment of human disorders and one of these ω-conotoxin MVIIA (ziconotide), an N-type calcium channel blocker, is currently used to treat pain in human patients by means of an implantable, programmable pump with a catheter threaded into the intrathecal space.

In certain embodiments of the present invention, the antiepileptic drug formulation comprises ω-conotoxins such as ω-conotoxin GVIA, ω-conotoxin MVIIA and ω-conotoxin CVID. See, e.g., Gasior et al. J. Pharmacol. Exp. Ther. 323:458-68 (2007). In alternative embodiments, the antiepileptic drug formulation comprises μ-conotoxins such as μ-conotoxin GIIIA, μ-conotoxin GIIIB, μ-conotoxin GIIIC, μ-conotoxin PIIIA, μ-conotoxin SmIIIA, μ-conotoxin KIIIA. See, e.g., Zhang et al., J. Biol. Chem. 282:30699-30706 (2007). Other embodiments utilize derivatives or pharmaceutically acceptable salts of the conotoxins, as described herein.

Botulinum Toxins

The anaerobic, gram positive bacterium Clostridium botulinum produces a potent polypeptide neurotoxin, botulinum toxin, which causes a neuroparalytic illness in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death.

Botulinum toxin type A is the most lethal natural biological agent known to man. About 50 picograms of botulinum toxin (purified neurotoxin complex) type A is a LD50 in mice. One unit (U) of botulinum toxin is defined as the LD50 upon intraperitoneal injection into female Swiss Webster mice weighing 18-20 grams each. Seven immunologically distinct botulinum neurotoxins have been characterized, these being respectively botulinum neurotoxin serotypes A, B, C1, D, E, F and G each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD50 for botulinum toxin type A. Botulinum toxins have been used for the treatment of neuromuscular disorders characterized by hyperactive skeletal muscles, such as blepharospasm, strabismus and hemifacial spasm. Non-type A botulinum toxin serotypes have a lower potency and/or a shorter duration of activity as compared to botulinum toxin type A.

Although all the botulinum toxins serotypes apparently inhibit release of the neurotransmitter at the neuromuscular junction, they do so by affecting different neurosecretory proteins and/or cleaving these proteins at different sites. For example, botulinum types A and E both cleave the 25 kiloDalton (kD) synaptosomal associated protein (SNAP-25), but they target different amino acid sequences within this protein. Botulinum toxin types B, D, F and G act on vesicle-associated protein (VAMP, also called synaptobrevin), with each serotype cleaving the protein at a different site. Finally, botulinum toxin type C1 has been shown to cleave both syntaxin and SNAP-25. These differences in mechanism of action may affect the relative potency and/or duration of action of the various botulinum toxin serotypes.

Regardless of serotype, the molecular mechanism of toxin intoxication appears to be similar and to involve at least three steps or stages. In the first step of the process, the toxin binds to the presynaptic membrane of the target neuron through a specific interaction between the H chain (heavy chain of molecular weight of about 100 kDa) and a cell surface receptor; the receptor is thought to be different for each type of botulinum toxin and for tetanus toxin. The carboxyl end segment of the H chain, Hc, targets the toxin to the cell surface. In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This last step is thought to be mediated by the amino end segment of the H chain, HN, which triggers a conformational change of the toxin in response to a pH of about 5.5 or lower. Endosomes are known to possess a proton pump which decreases intraendosomal pH. The conformational shift exposes hydrophobic residues in the toxin, which permits the toxin to embed itself in the endosomal membrane. The toxin then translocates through the endosomal membrane into the cytosol. The last step of the mechanism of botulinum toxin activity appears to involve reduction of the disulfide bond joining the H and L chain. The entire toxic activity of botulinum and tetanus toxins is contained in the L chain (light chain of molecular weight of about 50 kDa) of the holotoxin; the L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane tetanus neurotoxin, botulinum toxin/B/D/F, and/G cause degradation of synaptobrevin [also called vesicle-associated membrane protein (VAMP)], a synaptosomal membrane protein. Most of the VAMP present at the cytosolic surface of the synaptic vesicle is removed as a result of any one of these cleavage events. Each toxin specifically cleaves a different bond.

In vitro studies have indicated that botulinum toxin inhibits potassium induced release of various neurotransmitters from primary cell cultures and brain synaptosome preparations. Glutamate is the neurotransmitter responsible for the bulk of synaptic excitation in the brain, and it is believed to be integral to the generation and spread of seizure discharges. It has been reported that botulinum toxin inhibits the evoked release of glutamate in primary cultures of spinal cord neurons and that in brain synaptosome preparations botulinum toxin inhibits the release of glutamate and other neurotransmitters.

In embodiments of the present invention, the antiepileptic drug is botulinum toxin A or botulinum toxin B. In other embodiments, the toxin is a fragment or an analog of botulinum toxin A or botulinum toxin B that possesses biological activity of the parent toxins. In other embodiments, the toxins are modified to bind specifically to appropriate targets on brain neurons. In some embodiments, recombinant techniques are used to produce the clostridial neurotoxins or their fragments or analogs.

Conantokins

Also contemplated for use in the present invention are conantokins, including those described in U.S. Pat. Nos. 6,172,041 and 6,399,574, the disclosures of which are expressly incorporated by reference herein.

Toxin Derivatives

Examples of derivatives include polypeptides in which one or more Arg residue is substituted by Lys, ornithine, homoarginine, nor-Lys, N-methyl-Lys, N,N-dimethyl-Lys, N,N,N-trimethyl-Lys or any synthetic basic amino acid. The Lys residues may be substituted by Arg, ornithine, homoarginine, nor-Lys, or any synthetic basic amino acid. The Tyr residues may be substituted with meta-Tyr, ortho-Tyr, nor-Tyr, mono-halo-Tyr, di-halo-Tyr, O-sulpho-Tyr, O-phospho-Tyr, nitro-Tyr or any synthetic hydroxy containing amino acid. The Ser residues may be substituted with Thr or any synthetic hydroxylated amino acid. The Thr residues may be substituted with Ser or any synthetic hydroxylated amino acid. The Phe residues may be D or L, may be substituted at the ortho, meta, and/or para positions with a halogen or may be substituted with any synthetic aromatic amino acid. The Trp residues may be substituted with Trp (D), neo-Trp, 6-halo-Trp (D or L), preferably 6-halo, or any aromatic synthetic amino acid; and the Asn, Ser, Thr or Hyp residues may be glycosylated. The halogen may be iodo, chloro, fluoro or bromo; preferably iodo for halogen substituted-Tyr and bromo for halogen-substituted Trp. The Tyr residues may also be substituted with the 3-hydroxyl or 2-hydroxylisomers (meta-Tyr or ortho-Tyr, respectively) and corresponding O-sulpho- and O-phospho-derivatives. The acidic amino acid residues may be substituted with any synthetic acidic amino acid, e.g., tetrazolyl derivatives of Gly and Ala. The Leu may be substituted with Leu (D). The Glu residues may be substituted with Gla or Asp. The Gla residues may be substituted with Glu or Asp. The acidic amino acid residues may be substituted with any synthetic acidic amino acid, e.g. tetrazolyl derivatives of Gly and Ala. The N-terminal Gln may be substituted with pyro-glutamate (Z). The aliphatic amino acids may be substituted by synthetic derivatives bearing non-natural aliphatic branched or linear side chains CnH2n+2 up to and including n=8. The Met residues may be substituted with nor-leucine (Nle). The Cys residues may be in D or L configuration and may optionally be substituted with homocysteine (D or L). Basic residues in the backbone may be D or L configuration. The central Trp residue within the ┐-turn is preferably epimerized to the D-form.

Examples of synthetic aromatic amino acid include, but are not limited to, nitro-Phe, 4-substituted-Phe wherein the substituent is C1-C3 alkyl, carboxyl, hyrdroxymethyl, sulphomethyl, halo, phenyl, —CHO, —CN, —SO3H and —NHAc. Examples of synthetic hydroxy containing amino acid, include, but are not limited to, 4-hydroxymethyl-Phe, 4-hydroxyphenyl-Gly, 2,6-dimethyl-Tyr and 5-amino-Tyr. Examples of synthetic basic amino acids include, but are not limited to, N-1-(2-pyrazolinyl)-Arg, 2-(4-piperinyl)-Gly, 2-(4-piperinyl)-Ala, 2-[3-(2S)pyrrolininyl)]-Gly and 2-[3-(2S)pyrrolininyl)]-Ala. These and other synthetic basic amino acids, synthetic hydroxy containing amino acids or synthetic aromatic amino acids are described by RSP Amino Acids LLC (Shirley, Mass.). Examples of synthetic acid amino acids include those derivatives bearing acidic functionality, including carboxyl, phosphate, sulfonate and synthetic tetrazolyl derivatives.

Additional derivatives are peptides in which the Asn residues may be modified to contain an N-glycan and the Ser, Thr and Hyp residues may be modified to contain an O-glycan (e.g., g-N, g-S, g-T and g-Hyp). In accordance with the present invention, a glycan shall mean any N-, S- or O-linked mono-, di-, tri-, poly- or oligosaccharide that can be attached to any hydroxy, amino or thiol group of natural or modified amino acids by synthetic or enzymatic methodologies known in the art. The monosaccharides making up the glycan can include D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified, e.g., with one or more O-sulfate, O-phosphate, O-acetyl or acidic groups, such as sialic acid, including combinations thereof. The gylcan may also include similar polyhydroxy groups, such as D-penicillamine 2,5 and halogenated derivatives thereof or polypropylene glycol derivatives. The glycosidic linkage is β and 1:4 or 1:3, preferably 1:3. The linkage between the glycan and the amino acid may be α or β.

Derivatives also include peptides in which pairs of Cys residues may be replaced pairwise with isosteric lactam or ester-thioether replacements, such as Ser/(Glu or Asp), Lys/(Glu or Asp), Cys/(Glu or Asp) or Cys/Ala combinations. In addition, individual Cys residues may be replaced with homoCys, seleno-Cys or penicillamine, so that disulfide bridges may be formed between Cys-homoCys or Cys-penicillamine, or homoCys-penicillamine and the like. Other embodiments include truncations of the natural toxins and their derivatives. Additional embodiments are non-peptides that mimic the activity of the peptides.

### Antiepileptic Drug Formulations

In one embodiment, the antiepileptic drug may be delivered within liposomes suitable for CED, e.g., gadolinium-liposomes. Liposomes are phospholipid bilayers formed into spheres in the presence of water that can be made to incorporate a variety of agents. The use of liposomes provides two advantages. First, the distribution of liposomes delivered by CED is very well characterized and may aid in reliable distribution of the antiepileptic drug. Second, the presence of the MRI tracer may allow a precise definition of the brain volume perfused using real-time MRI. Previous studies have shown that CED infusion of gadolinium-tagged liposomes produce no neurological or behavioral toxicity in rats and cause no histologically-demonstrable tissue damage.

In one embodiment, the antiepileptic drug is mixed with one or more tracer molecules or conjugated with a tracer molecule that permits the distribution of the toxin to be assessed using an imaging modality such as magnetic resonance imaging (MRI), computed tomography (CT) or single-photon emission computed tomography (SPECT).

Accordingly, the liposomes may contain the toxin along with a suitable tracer molecule. For example, liposomes impregnated with an MRI tracer (e.g., gadolinium) may be used, and may be loaded with an antiepileptic drug described herein, e.g., botulinum neurotoxin B. Other nonlimiting examples of tracers include fluorescent markers incorporated into the liposome (which may help define the distribution volume through histological analysis). An alternative to convecting liposomes comprising both tracer and antiepileptic drug may be convecting a mixture of two types of liposomes, in which the bulk of the liposomes may be carrying the antiepileptic drug and in which the remaining liposomes may be impregnated with the tracer, e.g., gadolinium. It has been demonstrated that gadolinium-tagged liposomes can be used as a tracer in this fashion and that they convect in an identical fashion to liposomes carrying a payload. Use of an MRI tracer allows real-time MRI to control the delivery of anti-epileptic drug to the desired brain region, e.g., by adjusting the rate of infusion and volume delivered during the infusion to ensure that the antiepileptic drug is delivered to the required brain volume.

Other well-known methods may be used to assess the distribution, retention, and verify the safety of the antiepileptic drug solutions for treating neurological disorders associated with excessive neuronal excitability.

### Treatment

The present invention is also useful to prevent epileptogenesis (the development of epilepsy) in an animal that is susceptible due to a genetic predisposition or an environmental factor, such as an injury to the nervous system.

In a preferred embodiment, the apparatuses and methods described herein may be used to treat epilepsy in subjects who are candidates for epilepsy surgery. The most common surgical procedure for epilepsy is temporal lobectomy. In this procedure, the anteromedial temporal lobe is resected or there is a more limited removal of the underlying hippocampus and amygdala. Another common surgical procedure for epilepsy is focal neocortical resection, which is applied for focal seizures arising from extratemporal regions. About 5% of patients develop clinically significant complications from surgery and about 30% of patients treated with temporal lobectomy will still have seizures. The methods described herein are less invasive than surgical resection and associated with less frequent and less severe complications. It may therefore be preferred in many instances to resective surgery.

Prior to the implantation of the CED infusion catheter-pump device, the subject may undergo evaluation similar to that which would be used prior to epilepsy surgery to localize the anatomic location of the seizure focus. The subject may undergo routine scalp or scalp-sphenoidal EEG recordings, or long-term EEG recordings. Video-EEG monitoring may be used to correlate behavioral manifestations of seizures with abnormal electrophysiologic activity on the EEG. Subjects may ordinarily undergo a high-resolution MRI scan to identify structural lesions. Additionally, SPECT and positron emission tomography (PET) may also be used to identify anatomic regions that may contribute to the subjects seizure activity. Once the presumed location of the seizure onset is identified, additional studies, including neuropsychological testing and the intracarotid amobarbital test (Wada test) may be used to assess language and memory localization and to determine the possible functional consequences of interference with the functional activity of the epileptogenic region by CED toxin infusion. In some cases, the exact extent of the abnormal brain region may be determined by performing cortical mapping using subdural grid electrodes or by using depth electrodes. This involves electrophysiologic recordings and cortical stimulation of the awake patient to identify the extent of epileptiform disturbances and the function of the cortical regions in question.

### Administration

Antiepileptic drugs may be administered in therapeutically effective amounts that will be apparent to the skilled artisan by virtue of a beneficial therapeutic response and the dose need is expected to vary depending upon the size of the epileptic focus that requires treatment. For example, ω-conotoxin GVIA. and ω-Conotoxin MVIIA may be administered in a dose of about 0.005 nmol to about 0.5 nmol into a small epileptic focus of a mammal. In cases of larger epileptic foci, correspondingly larger amounts of the toxin is administered. In another example, botulinum toxin A and botulinum toxin B may be administered in a dose of about 1.0 ng to about 3.2 ng into a small epileptic focus of a mammal. In cases of larger epileptic foci, correspondingly larger amounts of the toxin is administered.

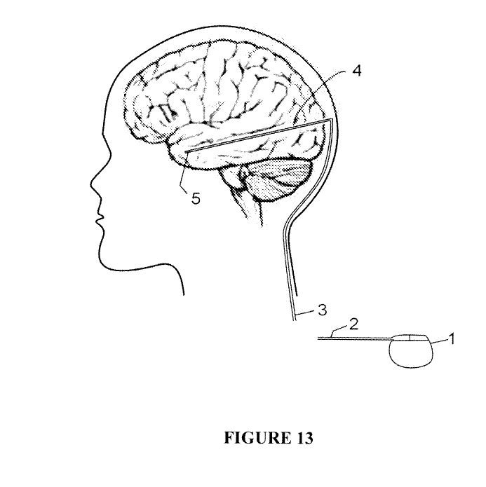

A schematic of one embodiment is shown in FIG. 13. In this embodiment, an apparatus employs an implantable pump (1) and a catheter, the catheter having a proximal end coupled to the pump (2) and a discharge portion (distal end; 5) that is placed within a predetermined infusion site in the brain for infusing therapeutic dosages of the toxin solution into the brain. The pump may be implanted in a subclavicular pocket and the intermediate portion of the catheter (3) may be tunneled under the skin of the neck and scalp around the surface of the skull and enters the skull through a burr hole. The catheter continues within the brain (4) so that the tip is situated in the brain region that requires treatment, most commonly in the temporal lobe as illustrated. The discharge portion of the catheter may consist of the open end of the catheter or there may be multiple discharge ports to allow a greater area of brain to be exposed to the infusion solution. It is recognized that the location, size and regional extent of the epileptic focus varies greatly among patients with partial epilepsy. Therefore, the region of brain infused must be tailored individually. The pump may operated to discharge a predetermined dosage of the antiepileptic drug solution through the discharge portion of the catheter into the infusion site. The antiepileptic drug solution may be infused at an appropriate rate and for an appropriate duration to convect the antiepileptic drug and distribute it within the epileptic focus or foci, e.g., the brain region responsible for the seizure activity. The antiepileptic drug may be reinfused at appropriate intervals. With each infusion, the epileptic seizures may be reduced for about one week to about 2 months, but seizure protection may last as long as 6 months or more per toxin infusion, depending upon the type of toxin and amount delivered.

Changes in detail or structure may be made without departing from the spirit of the invention as defined in the appended claims. Accordingly the matter contained in the above description or shown in the accompanying drawings shall be interpreted as illustrative only and not limiting.

### EXAMPLES

Described herein, it is shown that CED infusion of the N-type calcium channel antagonists ω-conotoxin GVIA (ω-CTX-G) and ω-conotoxin MVIIA (ω-CTX-M) can attenuate kindling measures in fully amygdala kindled rats. Rats were implanted with a combination infusion cannula-stimulating electrode assembly into the right basolateral amygdala. Fully kindled animals received infusions of vehicle, ω-CTX-G (0.005, 0.05, 0.5 nmol), ω-CTX-M (0.05, 0.15, 0.5 nmol), proteolytically inactivated ω-CTX-M (0.5 nmol), or carbamazepine (500 nmol) into the stimulation site. CED of ω-CTX-G and ω-CTX-M over a 20-min period resulted in a dose-dependent increase in the afterdischarge threshold and a decrease in the afterdischarge duration and behavioral seizure score and duration during a period of 20 min to 1 week after the infusion, indicating an inhibitory effect on the triggering and expression of kindled seizures. The protective effects of ω-conotoxins reached a maximum at 48 h post infusion and then gradually resolved over the next 5 days. In contrast, carbamazepine was active at 20 min but not at 24 h after the infusion, whereas CED of vehicle or inactivated ω-CTX-M had no effect. Except for transient tremor in some rats receiving the highest toxin doses, no adverse effects were observed. These results indicate that local CED of high molecular weight presynaptic N-type calcium channel blockers can produce long lasting inhibition of brain excitability and may provide prolonged seizure protection in focal seizure disorders.

### Example 1

### Materials and Methods

### Example 1.1

### Animals

Experimentally-naive, male Sprague-Dawley rats, weighing 225 to 250 g at the beginning of the study, were obtained from Taconic Farms, Germantown, N.Y. Rats were housed individually under a controlled environment (temperature, 24±2° C.; humidity, 45±5%; 12-hour light-dark cycle with lights on between 6:00 and 18:00 h). Each rat had free access to tap water and a nutritionally balanced rodent diet supplemented regularly with fresh fruit and sweetened gelatin dessert. Experiments were conducted between 9:00 and 16:00 h.

All experiments were conducted under a protocol approved by the Institutional Animal Care and Use Committee of the National Institute of Neurological Disorders and Stroke (NINDS) in strict compliance with the Guide for the Care and Use of Laboratory Animals of the National Research Council (National Academy Press, Washington, D.C.; http://www.nap.edu/readingroom/books/labrats/). The animal facilities were fully accredited by the American Association for the Accreditation of Laboratory Animal Care.

### Example 1.2

### Test Substances

Synthetic ω-CTX-G (27 amino acids; MW, 3037) and ω-CTX-M (25 amino acids; MW, 2639) were from Sigma-Aldrich (St. Louis, Mo.). Each toxin was dissolved in a 0.01% (w/v) solution of bovine serum albumin in 0.1 M phosphate buffered saline (PBS). Inactivated ω-CTX-M was obtained by mild treatment with chymotrypsin as described by Chung et al. (1995) Int J Pept Protein Res 46:320-325. Briefly, 4 μg of sequencing grade modified bovine chymotrypsin (Princeton Separations, Adelphia, N.J.) was added to a solution of 100 μg ω-CTX-M in 150 μL 0.1 M PBS. The solution was then incubated at 30° C. overnight. At the end of the incubation, the solution was boiled in a water bath for 60 min in order to inactivate the chymotrypsin, and then diluted with 0.1 M PBS to the final concentration of 0.5 nmoles/5 μL. Such an enzymatic digestion of ω-CTX-M results in the selective cleavage of the peptide link between tyrosine-13 and aspartic acid-14 and results in a 99% decrease in the toxin's binding affinity for neuronal N-type calcium channels (Nadasdi et al., 1995). Carbamazepine (Sigma-Aldrich) was dissolved in 30% (w/v) polyethylene glycol 400 in 0.1 M PBS.

### Example 1.3

### Kindling

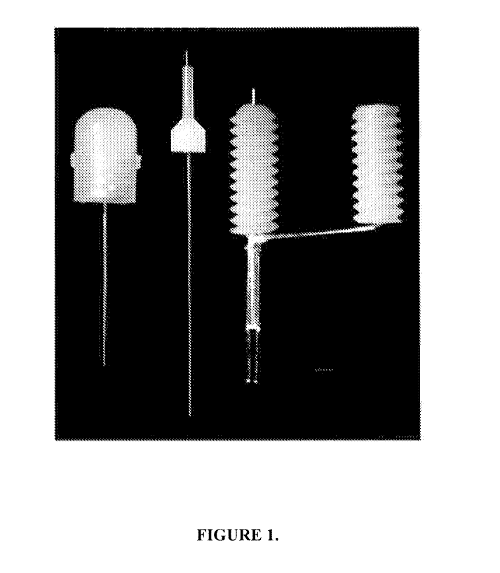

Each rat was chronically implanted with a custom-made guide cannula-bipolar stimulating electrode assembly (Part C315G-MS303/2; Plastics One, Roanoke, Va.; FIG. 1) during an aseptic surgical procedure under general anesthesia induced by a mixture of ketamine (60-75 mg/kg) and medetomidine (0.25-0.5 mg/kg). The cannula-electrode assembly consisted of a 26-gauge, 6.5-mm long stainless steel guide cannula with two 0.23 mm-diameter stainless steel electrode wires attached diametrically opposed at the periphery. The electrode wires were polyamide-insulated except for the 0.5 mm most distal extent; the tips of the wires were separated by 0.5 mm and projected 3.5 mm past the end of the guide cannula. The cannula-electrode was fixed to a threaded central plastic pedestal. The electrode wires passed to a second threaded pedestal with pin connectors to allow a connection with the kindling stimulator. The bipolar electrodes were used for recording and stimulating. Between infusions, the guide cannula was plugged with a dummy cannula assembly consisting of a solid wire of the same length (6.5-mm long) as the guide cannula and a threaded plastic cap (Part C315DC; Plastics One). The electrode tip was implanted into the basolateral nucleus of the right amygdala at stereotaxic coordinates (AP: −2.8 mm; ML: 5.0 mm; DV: −8.7 mm) measured from bregma (Paxinos G and Watson C (1998) The rat brain in stereotaxic coordinates, 4th ed. Academic Press, Sydney). Dental acrylic cement (Lang Dental, Wheeling, Ill.) and stabilizing stainless steel screws (Plastics One) were used to secure the cannula-electrode assembly to the skull. Ketoprofen was given subcutaneously after surgery, followed by atipamezole (1 mg/kg) to reverse anesthesia. At least ten days were allowed for recovery after the surgery. The position of the cannula-electrode assembly tip was histologically verified in randomly selected rats at the end of the study.

Rats were stimulated individually within a 29 cm diameter Plexiglas cylinder. Each rat was connected to a custom made stimulator (National Institutes of Health Research Services Branch, Bethesda, Md.) via a swivel attachment to allow free movement within the chamber. The stimulator was set to deliver 1 ms-duration, bipolar, square current pulses at 60 Hz for 1 s at variable current intensities. Depth electroencephalogram (EEG) signals were recorded via the stimulating electrode (except during the 1-s stimulation interval) with a Grass CP511 AC EEG preamplifier (Astro-Med, West Warwick, R.I.) and stored in digital form using Axotape 9 (Axon Instruments, Foster City, Calif.).

Kindled seizure activity was assessed using four dependent measures: afterdischarge (AD) threshold, AD duration, severity of behavioral seizures, and behavioral seizure duration. AD threshold refers to the lowest stimulating current intensity (in μA) that induces an AD consisting of a train of EEG spikes at 1 Hz or more lasting for at least 5-s duration with amplitude at least twice the baseline amplitude. AD duration is the total duration of the AD (in s). The severity of behavioral seizures was scored according to Racine with the following designations: stage 0, no apparent change in behavior; stage 1, facial twitching; stage 2, head nodding associated with more severe facial twitching; stage 3, unilateral forelimb clonus; stage 4, rearing; stage 5, rearing and loss of balance ((1972b) Electroencephalogr Clin Neurophysiol 32:281-294). Behavioral seizure duration is the duration (in s) of limbic seizures (stage 1-2) or motor seizures (stage 3-5). Behavioral changes such as immobility with occasional facial twitches that often occurred after the end of motor seizures were not considered in the duration determination.

After the rats recovered from the surgery, kindling began and consisted of three phases: (1) pre-kindling determination of the AD threshold; (2) kindling development, and (3) post-kindling redetermination of the AD threshold (Pinel, J. P., et al. (1976) Epilepsia 17:197-206; Freeman, F. G. and Jarvis, M. F. (1981) Brain Res. Bull. 7:629-33). On day 1, AD threshold was determined by delivering a series of stimulations of increasing intensities (starting at 50 μA and increasing in 25% increments every 3-5 min) until AD was triggered. Rats were excluded from the study if a current of 466 μA intensity failed to produce AD on the first day of kindling. (Rats excluded for this and other reasons, including headmount loss and guide cannula blockage, did not exceed 15% of animals.) During the second phase of kindling, each rat was stimulated daily at a current intensity of 125% of its individual AD threshold value determined on day 1. Daily stimulations continued until the rat exhibited stage 5 seizures during 5 consecutive days or on 8 days out of the last 10 stimulation days. Rats meeting this criterion were considered “kindled.” Rats that failed to meet the kindling criterion within 30 stimulations were excluded from the study. During the third phase of kindling, AD threshold was redetermined in the same way as during the first phase of kindling. Threshold was redetermined on several consecutive days until AD threshold and the behavioral seizure score produced by stimulation at the AD threshold were stable and reproducible. Rats that did not show stable responses to the electrical stimulation at their respective AD thresholds for several consecutive days were excluded from further testing.

### Example 1.4

### Liposomes

Liposomes are prepared to contain various combinations of a magnetic resonance (MR) contrast agent (gadodiamide or gadoteridol), a fluorescent marker (Dil-DS or sulforhodamine B), and an antiepileptic drug (e.g., botulinum neurotoxin B). The MR contrast agent is used to visualize the distribution of liposomes using MRI. The fluorescent marker is used to visualize the distribution of the liposomes histologically. The liposomes are made from 1-2-dioleoyl-3-sn-glycerophosphocholine and cholesterol (molar ratio, 3:2) and N-methoxy-poly(ethylene glycol)-1,2-distearoyl-3-sn-phosphoethanolamine (5 mol %), which yield liposomes ˜75-125 nm in diameter (as determined by dynamic light scattering). A Sephadex column is used to remove unincorporated gadolinium chelate. The liposome concentration is measured using a standard phosphate assay and adjusted to 20 mM phospholipid for all experiments. The concentration of gadolinium in the liposomes is determined by nuclear MR relaxivity measurements.

### Example 1.5

### Convection-Enhanced Delivery (CED)

The CED system consisted of a programmable infusion pump (model KDS200, KD Scientific Inc., Holliston, Mass.), a gas-tight 50 μL Hamilton syringe with a 22-gauge needle (Hamilton Company, Reno, Nev.), a counter-weighted swivel to allow free movement of the rat (Instech Laboratories Inc., Plymouth Meeting, Pa.), and a 33-gauge infusion cannula (part C315I; Plastics One). All components were connected with thick-wall polyethylene 50 tubing (Plastics One). After gently restraining the rat, the infusion cannula (FIG. 1) was slowly inserted into the brain through the guide cannula. The tip of the infusion cannula extended to a depth 0.5 mm above the tips of the stimulating electrode wires and was maintained at the appropriate depth by a plastic stop at the top of the cannula. The rat was released and placed in a plastic cylinder for the entire infusion. All infusions were performed in conscious and unrestrained animals. After infusion cannula insertion, the brain tissue was allowed to seal around the cannula for a few minutes before initiation of the infusion. Antiepileptic drug solution was delivered at a constant rate of 0.25 μL/min, which has previously been determined to be optimal for CED of large molecules into the rat brain (Chen et al., 1999). The total infusion volume was 5 μL. At the end of the infusion, the cannula was left in place for a few minutes to minimize antiepileptic drug solution back-flow and to ensure better distribution of antiepileptic drug solution. Experiments to assess the effects of bolus injection were carried out in an identical fashion except that the infusion rate was 2.5 μL/min.

Evaluation of the effects of test substances on kindling measures and for neurological toxicity. Effects of test substances on seizure sensitivity in fully-kindled rats were assessed by establishing the AD threshold as described previously and measuring the AD duration, seizure stage and behavioral seizure duration. Following CED infusion of the test substances, animals were stimulated and kindling measures were determined 20 min post-infusion as well as on the subsequent days at 24 h, 48 h, 72 h, 96 h and 1 week post-infusion. Each rat was observed for the occurrence of tremor (rhythmic oscillatory movements of the limbs, head and trunk) or other neurological signs during the test substance infusion, for at least 1 hour after the infusion, and before each subsequent stimulation session. In some animals, stimulation and determination of kindling measures was carried out only during the baseline period and at 7 and 8 days post-infusion.

The effects produced by the test substances on kindling measures were fully reversible within one week after infusion so that rats could be reinfused and retested, allowing the total number of animals used in the study to be minimized. Rats were reused for testing different toxin doses. Also, randomly selected rats from groups previously tested with toxins were reused for testing inactivated ω-CTX-M and carbamazepine. All infusions were separated by at least 14 days. To ensure that there were no interactions between the treatments, all four kindling measures were re-evaluated for several days prior to re-use and compared with the initial baseline values for that animal. Animals were reused only if the AD threshold value and other kindling measures determined upon re-evaluation were close to the baseline values for that animal.

### Example 1.6

### Locomotor Activity

At the end of the studies characterizing toxin effects on kindling measures, 18 fully-kindled rats were randomly selected for the locomotor activity testing with a VersaMax Animal Activity Monitoring System (AccuScan Instruments, Columbus, Ohio). The locomotor activity chamber consisted of a Plexiglas arena (40 cm×40 cm) surrounded by two sets of infrared light beam sensors detecting horizontal movement of 1.8 cm (at a level of 2.5 cm above the floor) and vertical movement 17.5 cm above the floor. The number of horizontal and vertical beam interruptions (reflecting ambulatory activity and rearing, respectively) were measured during daily sessions lasting 60 min each.

Each rat was exposed to the locomotor-activity chamber for 60 min on 5 successive days to allow habituation. Horizontal and vertical activity trended toward a stable baseline over the 5-day period; the means of the activity counts during the test session on the final two days of the habituation period were taken as the baseline for the infusion studies. On the day after the completion of the 5-day habituation period, each rat received an infusion of a test substance. The parameters of the infusion and other factors including animal handling and external cues were identical to those in the kindled seizure experiments. Horizontal and vertical beam interruptions were determined in 60 min periods beginning 20 min post infusion and on subsequent days at 24 h, 48 h, 72 h and 96 h post infusion.

### Example 1.7

### Intraventricular Infusion

Non-kindled rats for intraventricular infusion studies had a guide cannula assembly (without stimulating electrodes) implanted at stereotaxic coordinates with respect to bregma of AP: −0.80 mm; ML: 1.4 mm; DV: −2.6 mm. At the time of infusion, an infusion cannula was inserted through the guide cannula so that the tip extended 1 mm beyond the end of the guide cannula to a DV depth of −3.6 mm within the right lateral ventricle. The toxin infusion proceeded as for CED at a rate of 0.25 μL/min over 20 min.

### Example 1.8

### Histological Analysis

After the completion of testing, selected animals were perfused transcardially with 4% paraformaldehyde and the brains were removed for sectioning and cresyl violet and silver staining to assess cannula placement and evidence of neuronal damage.

### Example 1.9

### Presentation of Experimental Data and Statistical Analyses

Kindling measures and locomotor-activity test values are presented as group means±S.E.M. For kindling measures, baseline values (used to calculate percent change values) are the averages of values collected during at least three sessions before CED infusion. Averaged relative values with respect to the corresponding average baseline value are used to present the results in the CED infusion experiments. Specifically, effects of each drug treatment are expressed as a change (in percent) from baseline calculated using the following formula: 100×[(value before treatment)−(value after treatment)]/(value before treatment). Treatment effects with respect to baseline for each rat were calculated separately and then averaged for a group. Statistical analyses of the data from the kindling and locomotor-activity testing were performed by one-way (within a group) and two-way (between groups) repeated measures analysis of variance (ANOVA) after transformation of the percentage change data using arcsine-root transformation. When appropriate, post hoc analysis was performed using Dunnett's test or Tukey's test. Tremor data are expressed as frequencies analyzed by the Fisher's exact probability test.

All calculations were made using SigmaStat (SPSS, Chicago, Ill.) and GraphPad Prism (GraphPad Software, San Diego, Calif.). Group differences were considered statistically significant at p<0.05.

### Example 2

### Results

### Example 2.1

### Kindling

The data presented represent the results of experiments with 28 fully amygdala kindled rats. In these animals, daily stimulation at a current intensity of 125% of the AD threshold value resulted in the attainment of the criterion for full kindling (stage 5 seizures) in 15±1 (mean±S.E.M.) days. The mean AD threshold value in these animals was 197±24 μA on the first day of kindling. All animals in the test group exhibited stable thresholds to elicit stage 5 seizures during the redetermination period. The mean redetermination AD threshold was 96±8 μA and the mean AD duration was 95±4 s. Stimulations at the individual threshold value resulted in behavioral seizures of stage 4.8±0.1 that lasted for 89±3.5 s.

### Example 2.2

### Effects of CED of ω-CTX-G and ω-CTX-M on Kindling Measures

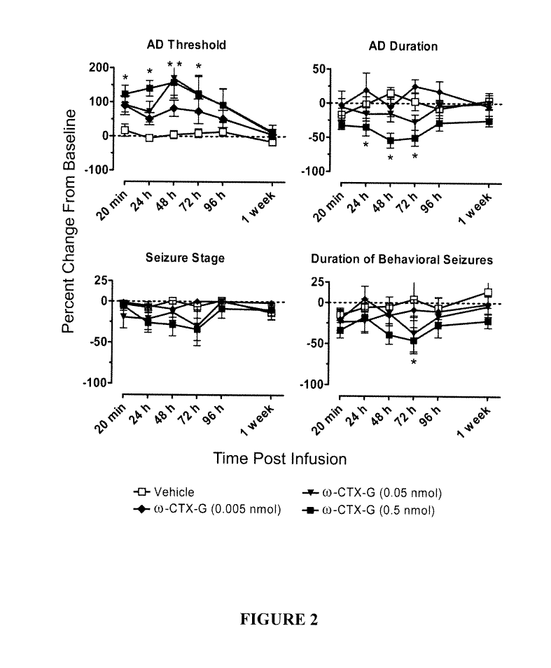

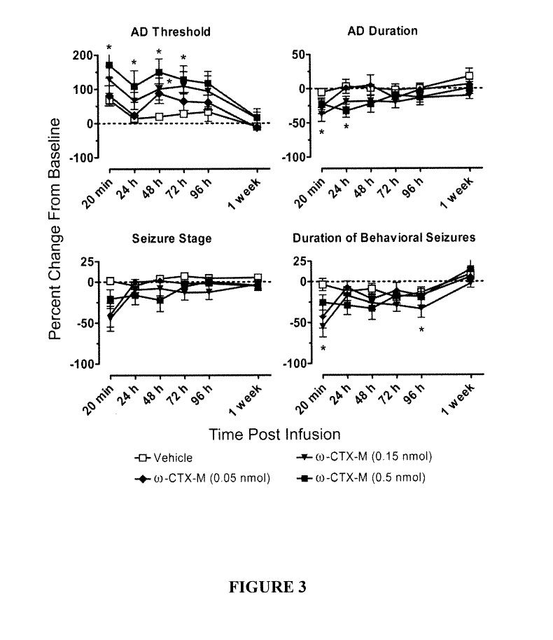

Animals receiving CED infusions of vehicle exhibited stable kindling measures (AD threshold, AD duration, seizure stage, and behavioral seizure duration) when tested 20 min after the infusion and at intervals up to 1 week (FIGS. 2 and 3). In contrast, a single 20 min CED infusion of ω-CTX-G (0.005-0.5 nmol) and ω-CTX-M (0.05-0.5 nmol) at time 0 was associated with an elevation in AD threshold at time points from 20 min to 96 h post infusion. In addition, there were effects of both toxins on AD duration, seizure stage and behavioral seizure duration. Two-way ANOVA indicated statistically significant treatment effects of ω-CTX-G on AD threshold (F_{3,101}=6.286, p=0.002), with accompanying decreases in AD duration (F_{3,101}=5.611, p=0.003) and behavioral seizure duration (F_{3,101}=3.049, p=0.041). ω-CTX-M was also associated with significant treatment effects on AD threshold (F_{3,124}=4.157, p=0.015), AD duration (F_{3,124}=3.498, p=0.028) and behavioral seizure duration (F_{3,124}=5.693, p=0.004). Post-hoc analyses indicated statistically significant effects of ω-CTX-G at doses of 0.05 and 0.5 nmol and of ω-CTX-M at doses of 0.15 and 0.5 nmol. The lowest doses tested of ω-CTX-G (0.005 nmol) and ω-CTX-M (0.05 nmol) were not associated with statistically significant effects on any of these measures. Finally, none of the doses tested of either toxin were associated with effects on seizure stage. Table 1 summarizes the results of the statistical analyses.

TABLE 1
Summary of the outcomes of two-way repeated measures
ANOVA and post-hoc statistical analyses (Tukey test)
for ω-CTX-G and ω-CTX-M effects on kindling
measures in the experiments of FIGS. 2 and 3.

| | ω-CTX-G | | | | ω-CTX-M | | | |
|---|---|---|---|---|---|---|---|---|
| | Treat- | Post-hoc analysis | | | Treat- | Post-hoc analysis | | |
| | ment | at dose (nmol) | | | ment | at dose (nmol) | | |
| | effect | 0.005 | 0.05 | 0.5 | effect | 0.05 | 0.15 | 0.5 |
| AD | 0.002 | NS | <0.05 | <0.05 | 0.015 | NS | <0.05 | <0.05 |
| threshold | | | | | | | | |
| AD | 0.003 | NS | NS | <0.05 | 0.028 | NS | <0.05 | NS |
| duration | | | | | | | | |
| Seizure | NS | | | | NS | | | |
| stage | | | | | | | | |
| Duration | 0.041 | NS | NS | <0.05 | 0.004 | NS | <0.05 | NS |
| of | | | | | | | | |
| behavioral | | | | | | | | |
| seizure | | | | | | | | |

Numerical values represent p values; NS, statistically insignificant (p > 0.05).

The effects on kindling measures in the experiments with ω-CTX-G and ω-CTX-M were time-dependent (FIGS. 2 and 3). The AD threshold was elevated at all time points from 20 min to 72 h after CED infusion of the highest does tested (0.5 nmol) of ω-CTX-G and ω-CTX-M. At 96 h the threshold elevations were smaller and not significantly different from the corresponding control values. The threshold values had nearly returned to the baseline pre-CED infusion values at 1 week. The two toxins at the highest doses produced effects of similar magnitude, with elevations in the AD threshold ranging from 106% to 153% with ω-CTX-G and 95% to 131% with ω-CTX-M at post-infusion intervals between 20 min and 72 h. Mean increases in the AD threshold during the same time period after CED infusion of the intermediate doses (0.05 nmol and 0.15 nmol, respectively) of ω-CTX-G and ω-CTX-M ranged from 75% to 164% and from 52% to 81%, with the maximum increase at 48 h following the CED infusion. The changes produced by the lowest toxin dose (0.005 nmol ω-CTX-G and 0.05 nmol ω-CTX-M) were not statistically significant.

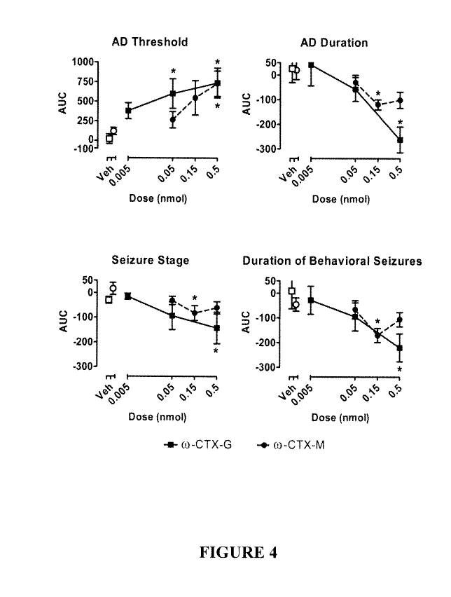

Analyses of the results of the results presented in FIGS. 2 and 3 by an area-under-the-curve method are presented in FIG. 4. ω-CTX-G exhibited statistically significant, dose-dependent effects on all kindling measures (AD threshold: F_{3,39}=5.05, p=0.007; AD duration: F_{3,39}=6.565, p=0.002; seizure stage: F_{3,39}=3.866, p=0.020; behavioral seizure duration: F_{3,39}=4.466, p=0.011). Similarly, ω-CTX-M also showed statistically significant treatment effects with respect to all kindling measures (AD threshold: F_{3,45}=3.305, p=0.033; AD duration: F_{3,45}=3.824, p=0.019; seizure stage: F_{3,45}=3.125, p=0.040; behavioral seizure duration: F_{3,45}=3.651, p=0.023). While the effect on AD threshold was dose-dependent for ω-CTX-M, dose-dependence for the 0.15 and 0.5 doses was not obtained for the other kindling measures.

The highest doses of ω-CTX-G and ω-CTX-M were associated with transient whole body tremor. In rats treated with ω-CTX-G (0.5 nmol), tremor was evident in 3 of 10 rats within 20 min following the CED infusion and in 9 of 10 rats 1 day after the infusion. In each of the animals that exhibited tremor, the tremor resolved by the third day after the infusion. The 0.5 nmol dose of ω-CTX-M was associated with tremor in 4 of 10 rats immediately after the infusion, but no tremor was apparent on subsequent days in any of the animals. No amygdala depth EEG abnormalities were detected in animals exhibiting tremor indicating that the motor behavior probably did not represent seizure activity. No tremor was observed after infusions of lower doses of the toxins or vehicle. Other than tremor, the CED infusions were not associated with any observable effects on neurological function or behavior.

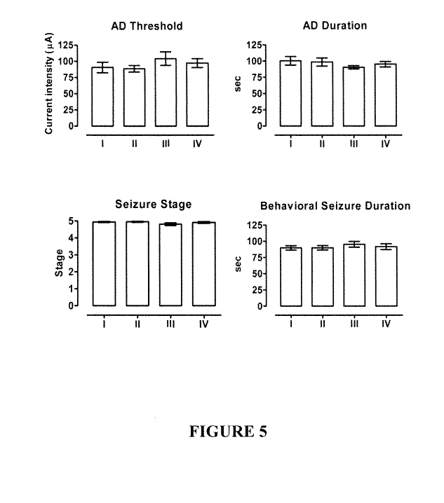

In order to minimize the use of animals, kindled rats were reused for the testing of different toxin doses and vehicle. Thus, each animal received at least four CED infusions (3 doses plus vehicle). During this sequence, the animals received multiple kindling stimulations. To verify that the baseline was stable throughout, mean baseline kindling measures for the complete group of 22 rats determined before each of the four sequential CED infusions were compared. As shown in FIG. 5, the baseline values in the kindling experiments were stable during the course of repeated infusion and testing (AD threshold: F_{3,63}=1.533, p=0.215; AD duration: F_{3,63}=1.078, p=0.365; seizure stage: F_{3,63}=1.501, p=0.233; behavioral seizure duration: F_{3,63}=0.620, p=0.604). Thus, it is unlikely that a history of previous testing had an influence on the response to subsequent testing.

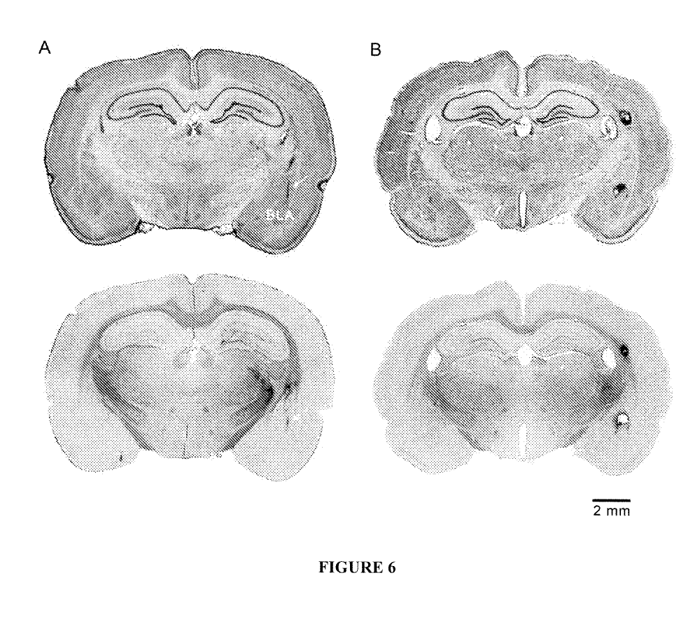

Histological examination of the brains from 5 animals tested with full sequences of toxin and vehicle demonstrated that multiple CED episodes were only associated with minimal damage to the amygdala and surrounding brain structures at the site of the infusion cannula-stimulating electrode assembly (FIG. 6A). An additional 8 rats received bolus infusions of 0.05 nmol ω-CTX-G in the same volume (5 μL) as for CED but at a 10-fold higher infusion rate (2.5 μL/min) using identical implanted infusion cannula-stimulating electrode assemblies. As in the example of FIG. 6B, 7 of these animals exhibited cavitation at the infusion site. The brain of the remaining animal was damaged during removal of the electrode assembly and was not be processed for histology

### Example 2.3

### Does Continued Kindling Account for the Apparent Recovery from the Toxin Effects?

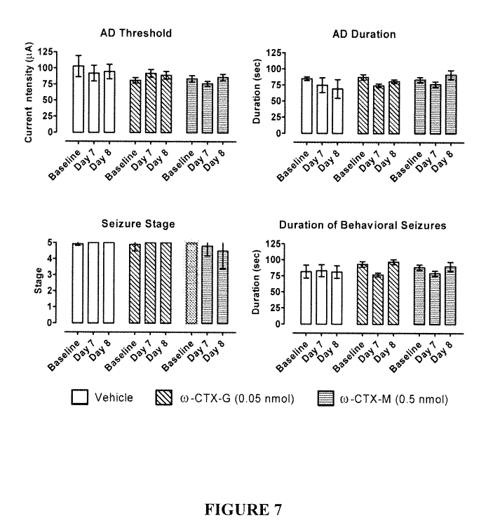

It is possible that the apparent recovery from the effects of toxin exposure over the 1 week period following toxin infusion in the experiments of FIGS. 2 and 3 was due to continued kindling resulting from repeated test stimulations and not to a decrease of the toxin effects. In this case, toxin effects on the kindling measures would be expected to be observed at time points beyond the apparent recovery period, if kindling stimulation is not imposed during the intervening period. To determine if such persistent effects of the toxin do occur, a subset of animals received CED infusion of ω-CX-G (0.05 nmol) and ω-CTX-M (0.5 nmol) but did not receive kindling test stimulation until day 7 after the infusion. As shown in FIG. 7, in these animals the kindling measures to test stimulations at days 7 and 8 were no different from the baseline values (p>0.05). Thus, the apparent recovery of the toxin effects in the experiments of FIGS. 2 and 3 was not likely to be due to repeated electrical stimulation.

### Example 2.4

### Lack of Effect of Proteolyzed ω-CTX-M on Kindling Measures

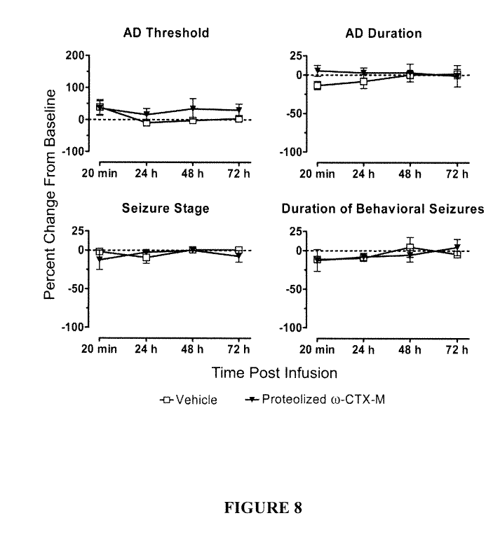

In order to determine whether the observed effects of the toxins were due to their activity as calcium channel antagonists, control experiments with proteolyzed ω-CTX-M which is devoid of effects on calcium channels were conducted. As shown in FIG. 8, CED infusion of proteolyzed ω-CTX-M (0.5 nmol) failed to cause a significant alteration in any of the kindling measures (AD threshold: F_{1,21}=0.0882, p=0.379; AD duration: F_{1,21}=1.115, p=0.318; seizure stage: F_{1,21}=0.486, p=0.508; and behavioral seizure duration: F_{1,21}=0.0007, p=0.981).

### Example 2.5

### Effects of Carbamazepine on Kindling Measures

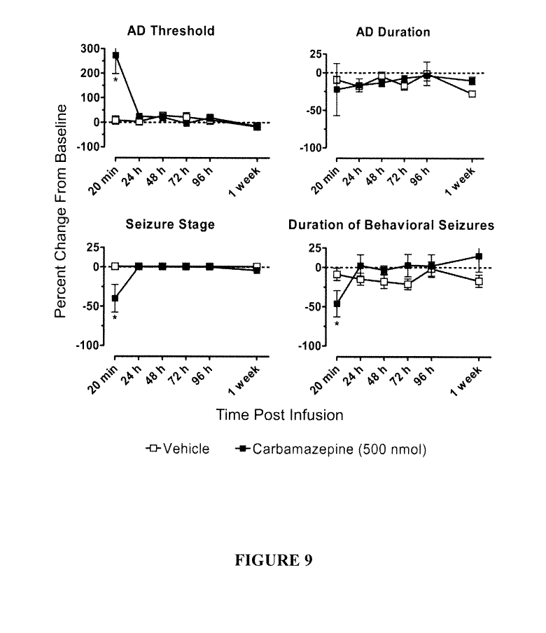

In order to determine if CED administration of a conventional non-peptide antiepileptic drug would produce long lasting effects on seizure measures like the toxins, a series of experiments with the widely used antiepileptic drug carbamazepine were conducted. CED infusion of carbamazepine (500 nmol) resulted in a significant increase in the mean AD threshold and decreases in mean seizure stage and mean behavioral seizure duration at the 20 min time point only (p<0.05) (FIG. 9). There were no differences in kindling measures at any of the later time points (24 h to 1 week). Thus, by comparison, CED administration of the toxins is associated with a distinctively long duration of action. Carbamazepine infusion was not associated with any observable adverse neurological or behavioral effects.

### Example 2.6

### Effects of CED Infusion of ω-CTX-G and ω-CTX-M on Locomotor Activity

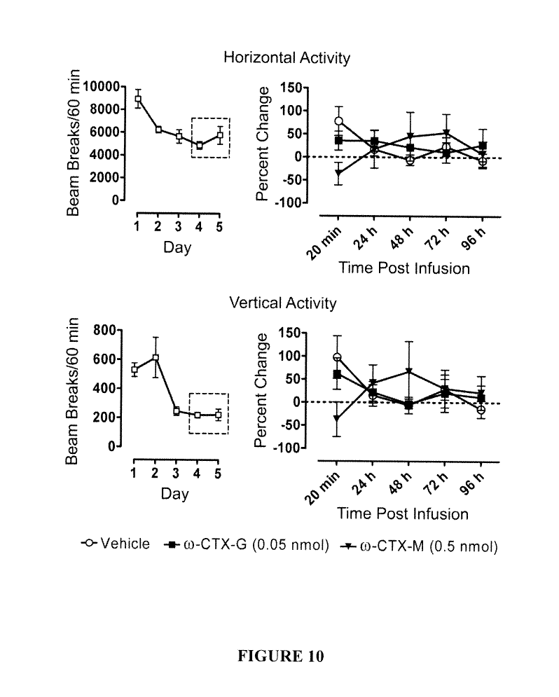

Animals selected for testing were habituated for five consecutive days to the locomotor activity chamber while recording horizontal and vertical activity. As FIG. 10 shows, the rats quickly habituated to the new environment as reflected by stable, low level locomotor activity after several 60-min habituation sessions. Habituated animals received CED infusions of ω-CTX-G (0.05 nmol), ω-CTX-M (0.5 nmol) or vehicle. The toxin doses chosen were the highest doses that produced significant effects on kindling measures but did not cause more than transient tremor. Locomotor activity was recorded in five 60-min sessions at intervals following the infusion. Nether toxin was associated with a change in horizontal activity (F_{2,60}=0.0413, p=0.960) or vertical activity (F_{2,60}=0.0021, p=0.998) in comparison with vehicle.

### Example 2.7

### Behavioral Effects of Intraventricular ω-CTX-G

To determine if CED infusion provided reduced toxicity compared to conventional intraventricular delivery, ω-CTX-G was administered intraventricularly and observed the animals for gross behavioral disturbances. All six rats injected intraventricularly with 0.05 nmol ω-CTX-G exhibited locomotor activity arrest [beginning 5.0±0.4 min (mean±S.E.M.) following the infusion] followed by rhythmic whole body tremors (beginning 10.8±0.9 min following the infusion). Calculated from the infusion rate and time from infusion onset, the cumulative doses corresponding to the thresholds for locomotor arrest and whole body tremors were 0.013±0.001 and 0.027±0.002 nmol, respectively. The full dose of toxin was associated with rapid, rhythmic shaking of the entire body. The forelimbs and hindlimbs were abducted so that the animal was unable to stand, causing it to rest on its abdomen. Rats experiencing tremor did not engage in normal behaviors, such as cage exploration, ambulation, eating or drinking. Consequently, there was significant body weight loss (mean±S.E.M.: 10.3±0.9%) at 24 h compared with the animals that had received the same dose of toxin by CED (body weight increase at 24 h, 0.1±2.1%; p<0.001 by t-test). Tremors persisted for 24 h, when the animals were euthanized for animal welfare concerns. The overall behavioral syndrome associated with intraventricular infusion of 0.05 nmol ω-CTX-G was much more severe than that observed after CED infusion of 0.5 nmol ω-CTX-G. In the latter instance, tremor was more modest in intensity, usually only evident during handling, and not associated with an interruption of normal behaviors such as exploration or feeding.

Three additional rats were injected intraventricularly with a 0.005 nmol dose of ω-CTX-G, which is below the previously determined threshold for intraventricular toxicity. Accordingly, these animals exhibited no or only mild behavioral effects of the toxin infusion. One of these animals did not exhibit any locomotor disruption or tremor. The second animal showed transient locomotor disruption at the end of the 20 min infusion and no tremor during the entire 24 h observation period. The third rat did exhibit transient locomotor activity disruption and mild, transient tremor during the infusion; both signs had resolved at 24 h. On average, these 3 rats showed a body weight gain of 3.0±1.8% at 24 h.

### Example 3

### Use of Botulinum Toxins as Antiepileptic Drugs Administered Via CED

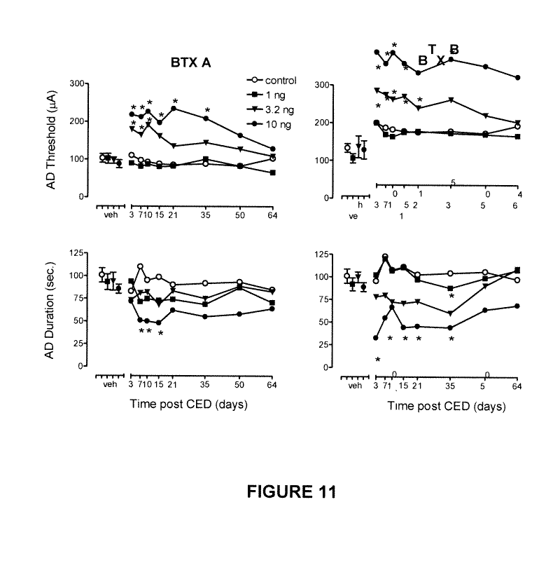

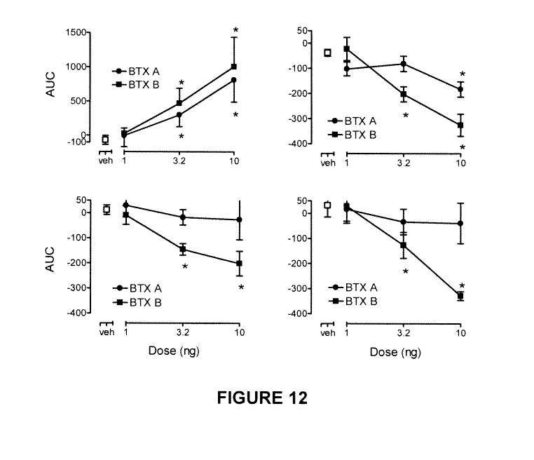

An extensive set of experiments to evaluate the ability of botulium toxin A and boulinum toxin B delivered by CED to protect against seizures in the rat kindling model were also performed. Briefly, rats were implanted with a combination guide cannulas-stimulation electrode into the right basolateral amygdala. Daily kindling stimulation was performed until each rat was fully kindled (stage 5 seizures for at least five consecutive days). Then, the rats received CED infusions of botulinum neurotoxin A (BTX-A; 1-10 ng) or botulinum neurotoxin B (BTX-B; 3.2-10 ng) into the stimulation site over 20 min. Each dose was delivered in a volume of 5 μL at a rate of 0.25 μL/min. Depth EEG (afterdischarge threshold and duration) and behavioral (seizure stage and duration) measures of amygdala-kindled seizures were recorded up to 64 d after the infusion. As illustrated in FIG. 11, 20 min CED infusion of both toxins caused a prolonged elevation in kindling parameters. This effect occurred in a dose-dependent fashion, where 1 ng of toxin was inactive, 3.2 ng caused an intermediate effect and 10 ng caused a more marked effect (see FIG. 12). It is notable that the duration of action was very prolonged. Botulinum toxin A had an effect that lasted more than one month, whereas botulinum toxin B lasted more than two months. Both toxins demonstrated reversibility although the duration of the experiments (64 days) was not sufficiently long to observe complete recovery with botulinum toxin B. Infusions of both toxins were well tolerated and were not associated with any immediate or long-term behavioral sequelae, confirming prior studies have demonstrated that CED is not associated with any tissue damage or neurotoxicity, apart from the local damage caused by the infusion cannula itself (Gasior, M. et al. (2007) J. Pharmacol. Exp. Ther. 323:458-468). Botulinum toxin B appeared to be more efficacious as determined by effects on behavioral seizure scores (Table 2).

TABLE 2
Summary of the outcomes of two-way repeated measures
ANOVA and post-hoc statistical analyses (Tukey test)
for botulinum neurotoxin A and botulinum neurotoxin
B effects on kindling parameters.

| | Botulinum Neurotoxin A | | | | Botulinum Neurotoxin B | | | |
|---|---|---|---|---|---|---|---|---|
| | Treat- | | | | Treat- | | | |
| | ment | Post-hoc analysis | | | ment | Post-hoc analysis | | |
| | effect | at dose (ng) | | | effect | at dose (ng) | | |
| | (F_{3, 175}) | 1.0 | 3.2 | 10 | (F_{3, 175}) | 1.0 | 3.2 | 10 |
| AD | 0.005 | NS | <0.05 | <0.05 | 0.009 | NS | <0.05 | <0.05 |
| threshold | | | | | | | | |
| AD | 0.008 | NS | NS | <0.05 | <0.001 | NS | <0.05 | <0.05 |
| duration | | | | | | | | |
| Seizure | NS (0.469) | | | | <0.001 | NS | <0.05 | <0.05 |
| stage | | | | | | | | |
| Duration | NS (0.725) | | | | <0.001 | NS | <0.05 | <0.05 |
| of | | | | | | | | |
| behavioral | | | | | | | | |
| seizure | | | | | | | | |

Numerical values represent p values; NS, statistically insignificant (p > 0.05).

### Example 4

### Discussion

ω-CTX-G and ω-CTX-M are peptide toxins originally isolated from the venoms of fish-hunting cone snails (Olivera et al. (1985) supra; Olivera, B. M., et al. (1987) Biochemistry 26:2086-90). The toxins selectively inhibit N-type voltage-activated calcium channels (Caᵥ2.2; Dubel, S. J., et al. (1992) Proc. Natl. Acad. Sci. USA 89:5058-62). At central synapses, multiple calcium channel types, including N-type calcium channels, mediate the influx of calcium into presynaptic nerve terminals that is required for neurotransmitter release (Wu, L. G. and Saggau, P. (1997) Trends Neurosci. 20:204-12). By depressing N-type calcium currents in presynaptic terminals, ω-CTX-G and ω-CTX-M inhibit both excitatory and inhibitory synaptic transmission (Kamiya et al. (1988) supra; Dutar et al. (1989) supra; Horne, A. L. and Kemp, J. A. (1991) Br. J. Pharmacol. 103:1733-1739; Burke, S. P. (1993) Eur. J. Pharmacol. 238:383-386; Luebke, J. I. (1993) Neuron 11:895-902). As a result of the effects on calcium channels, the toxins can inhibit epileptiform activity in some in vitro preparations (Boulton and O'Shaughnessy (1991) supra). Experiments disclosed herein determined whether ω-CTX-G and ω-CTX-M infused using the CED method can interfere with the electrical activation of kindled seizures and their subsequent expression electrographically and behaviorally. The results demonstrate that local CED administration of both toxins at the site of kindling stimulation can produce a prolonged increase in the intensity of electrical stimulation required to activate an electrographic seizure and can depress the seizures elicited.

A key result disclosed herein is that the toxins can produce long lasting effects on electrical excitability that persist for up to 1 week. The persistent action of the toxins contrasts with the duration of the response to carbamazepine, whose effect on stimulation threshold and on stimulation-induced seizures was apparent at 20 min but not at 24 h. The persistent action of ω-conotoxins has not previously been described. In a study of the analgesic effects of 7-day infusion of ω-CTX-M, Malmberg and Yaksh found that the action of the toxin had resolved when tested after stopping the infusion (Malmberg A B and Yaksh T L (1995) Pain 60:83-90). Two factors could account for the prolonged duration of action of the toxins in these experiments. The binding of ω-CTX-G and ω-CTX-M to N-type calcium channels under ordinary condition is extremely stable and is generally considered to be irreversible (Cruz, L. J. and Olivera, B. M. (1986) J. Biol. Chem. 261:6230-33; Grantham, C. J., et al. (1994) Neuropharmacology 33:255-58; Fox, J. A. (1995) Pflügers Arch 429:873-75). If the turnover of N-type calcium channels is low (or if toxin binding inhibits turnover), then the irreversible nature of toxin binding could account for the persistent blocking action of the toxin. In this case, recovery would result from resynthesis of new channel protein. Little is known about the stability of N-type calcium channels at central nervous system synapses. The results disclosed herein are compatible with the possibility that channel turnover is slow and occurs over the course of days. Applicants note that under this scenario the channel turnover rate may not necessarily be the dominant rate accounting for recovery from the toxin effects inasmuch as dissociation of toxin from N-type calcium channels, while slow, could nevertheless occur over the time course of days. The second factor that could contribute to prolonged toxin action is the inability of toxin molecules to leave the site of deposition in the extracellular space following CED infusion. Carbamazepine is a strongly hydrophobic molecule (log P of 2.69 by XLogP; Wang, R. et al. (1997) J. Chem. Inf. Comput. Sci. 37:615-21) which readily diffuses across biological membranes. Thus, it can easily leave the site of deposition. Accordingly, even though a relatively large amount of carbamazepine was delivered in the CED experiments, its effects dissipated rapidly. By contrast, ω-CTX-G and ω-CTX-M are hydrophilic [grand average of hydropathicity (GRAVY) values, −0.893 and −0.464, respectively; Kyte, J. and Doolittle, R. F. (1982) J. Mol. Biol. 157:105-32] and would be expected to remain in the extracellular space at the site of deposition. Although the toxins are susceptible to cleavage by endopeptidases and exopeptidases at multiple peptide bonds, cerebrospinal fluid exhibits minimal hydrolytic activity for the peptides, so that local degradation is not expected to limit the duration of the functional activity. The fact that a prolonged duration of action of the toxins when they were administered by CED was observed whereas the duration was not prolonged with instillation into the cerebrospinal fluid in the study of Malmberg and Yaksh (1995) suggests that slow toxin clearance may be of greater importance than slow dissociation from the target site as a determinant of the long duration of action of the toxins.

In binding studies, ω-CTX-G and ω-CTX-M have been shown to compete for the same target in brain, which represents the N-type calcium channel α1B subunit (Caᵥ2.2) (Feng, Z. P., et al. (2003) J. Biol. Chem. 278:20171-78). The kinetics of binding and affinity of the two toxins for expressed recombinant rat Caᵥ2.2 calcium channel subunits is similar (KD ˜100 nM). Mutagenesis studies have indicated that the two toxins bind to overlapping domains on the channel, but the recognition sites do not share identical structural determinants (id.; Nielsen, K. J. (2000) J. Mol. Recognit. 13:55-70). The efficacy and time-course of action of the toxins were similar, except that ω-CTX-G produced a significant effect on AD threshold at the 0.05 nmol dose whereas the effects of ω-CTX-M at this dose did not reach significance (Table 1). In a study with audiogenic seizure susceptible mice, intraventricularly administered ω-CTX-G protected against seizures whereas ω-CTX-M did not (Jackson and Scheideler (1996) supra), indicating that ω-CTX-G may have greater anticonvulsant potency than ω-CTX-M. However, upon intraventricular administration, both toxins induced intense shaking at very low doses. It was confirmed that intraventricular infusion of 0.05 nmol ω-CTX-G, a dose that was devoid of any gross behavioral effects when delivered by CED, caused profound behavioral disruption, including persistent whole-body tremor. Thus, untargeted administration of the conotoxins by instillation into the cerebrospinal fluid is not likely to be a useful therapeutic approach. In contrast, the results described herein indicate that local CED delivery of the toxins can protect against seizures with reduced or no untoward neurological side effects, highlighting the potential of local CED delivery for epilepsy therapy.

To confirm that the effects of toxin infusion were due to functional blockade of N-type calcium channels, it was demonstrated that CED infusion of proteolyzed ω-CTX-M, which has less than 1% of the binding affinity of the native toxin and is inactive physiologically (Nadasdi, L., et al. (1995) Biochemistry 34:8076-81), had no effect on kindling measures. In an additional series of control experiments, superkindling was excluded as an explanation for the apparent recovery from the toxin effects over the 1-week course of the CED studies. Superkindling is a phenomenon whereby continued daily kindling stimulations cause a progressive decrease in the AD threshold, presumably due to continuing kindling (Racine, R. J. (1972a) Electroencephalogr Clin. Neurophysiol. 32:269-79). Superkindling could lead to an apparent diminution in activity of the toxin. However, in experiments kindling stimulations were not applied until the 7th day after the toxin infusion, no change in any kindling measures on days 7 and 8 were found indicating that the toxin effect decreases even when kindling stimulations are not applied.

Although toxin infusion produced a remarkably long-lasting effect on kindling measures, there was no evidence that the repeated CED infusions or episodes of toxin exposure caused functionally significant tissue damage. Histological examination after several cycles of toxin infusion and multiple kindling stimulations failed to detect any evidence of tissue damage at sites beyond the tract caused by the physical placement of the infusion cannula-stimulating electrode assembly. In addition, as shown in FIG. 4, baseline kindling measures were stable over multiple CED infusions of vehicle and toxins at different doses. Thus, repeated CED infusion of the toxins did not produce permanent alterations in the electrical excitability properties of the brain at the infusion site. Moreover, there was no indication that the toxins had antiepileptogenic properties to reverse the epileptic state generated by the initial kindling stimulations.

Carbamazepine was highly protective when administered by CED infusion, consistent with its well-recognized activity against amygdala kindled seizures in rats when administered systemically (Albertson, T. E. et al. (1984) Neuropharmacology 23:1117-23). The peak action of carbamazepine occurred earlier than the toxins and its duration was dramatically shorter. The more rapid onset and recovery from carbamazepine are likely due to its ability to diffuse to the target within the infusion zone and then leave the infusion zone, as discussed above. Indeed, carbamazepine has a short duration of action when it is administered systemically or by the intracerebroventricular route in amygdala-kindled rats (Albertson et al. (1984) supra; Barcia, J. A., et al. (1999) Epilepsy Res. 33:159-67). Although the minimum effective dose of carbamazepine was not determined, carbamazepine appears to be markedly less potent than the toxins. The high potency of the ω-conotoxins might be advantageous in a clinical application in which an implanted pump of limited capacity is used to deliver the therapeutic agents.

ω-CTX-G and ω-CTX-M are well recognized to cause “shaking” when injected intracerebroventricularly in mice and, in fact, were initially isolated from cone snail venom on the basis of this activity (Olivera et al., (1985) supra; Miljanich and Ramachandran, (1995) supra). In the CED experiments disclosed herein, the highest doses of ω-CTX-G and ω-CTX-M were associated with tremor in some animals, but lower doses, even those that had long lasting effects on kindling measures, did not cause tremor. Thus, in contrast to the situation when the toxins are instilled into the cerebrospinal fluid, when administered by CED tremor can be dissociated from therapeutic activity. There were no other observable neurobehavioral effects of the CED toxin infusions. Specifically, the toxins did not cause any significant changes in locomotor activity during the 96 h period after CED infusion. ω-CTX-G was associated with more prolonged tremor than ω-CTX-M, further supporting the view that it is modestly more potent. Since toxin effects on kindling measures could be obtained in the absence of tremor, it was presumed that the tremor obtained with high toxin doses is due to leakage of the toxins from the amygdala infusion site. Such leakage is likely to be a particular problem in animals with small brains inasmuch as the distribution of substances administered by CED can be more effectively controlled in larger brain volumes (Lieberman, D. M., et al. (1995) J. Neurosurg. 82:1021-29; Lonser, R. R., et al. (2002) J. Neurosurg. 97:905-13).

Disclosed herein are what Applicants believe are the first experimental evidence that brief (20-min), localized CED infusions of ω-CTX-G and ω-CTX-M can produce prolonged alterations in brain excitability at the site of the infusion that persist for up to 1 week. Moreover, the results raise the possibility that the CED technique could have utility in suppressing focal seizures. Although ω-conotoxins were used to demonstrate the potential of the CED method, CED is equally applicable to other large blood-brain barrier impermeable antiepileptic drugs or biological agents, including other anticonvulsant peptides, and gene therapy vectors encoding proteins having anticonvulsant properties and/or that diminish or inhibit epileptiform discharges, e.g., neuropeptide Y, galanin, etc.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. In addition, to the extent that the prior art knowledge has not been explicitly incorporated by reference herein above, it is explicitly being incorporated herein in its entirety. All publications referenced above are incorporated herein by reference in their entireties.

What is claimed is:

1. A method of treating a neurological disorder associated with excessive neuronal excitability comprising administering to a subject in need of such treatment a antiepileptic drug solution comprising a therapeutically effective amount of an antiepileptic drug using convection-enhanced delivery (CED).

2. The method of claim 1, wherein the neurological disorder is a type of epilepsy.

3. The method of claim 2, wherein the type of epilepsy is selected from the group consisting of partial epilepsy, simple partial seizures, Jacksonian seizures, and complex partial (psychomotor) seizures.

4. The method of claim 1, wherein the antiepileptic drug is selected from the group consisting of a toxin that inhibits the exocytosis of neurotransmitters, and an excitotoxin.

5. The method of claim 4, wherein the antiepileptic drug is a toxin that inhibits the exocytosis of neurotransmitters.

6. The method of claim 5, wherein the toxin that inhibits the exocytosis of neurotransmitters is selected from the group consisting of a conotoxin and a botulinum toxin.

7. The method of claim 6, wherein the toxin that inhibits the exocytosis of neurotransmitters is a conotoxin.

8. The method of claim 6, wherein the conotoxin is selected from the group consisting of ω-conotoxin MVIIA and ω-conotoxin GVIA.

9. The method of claim 6, wherein the conotoxin is ω-conotoxin MVIIA.

10. The method of claim 6, wherein the conotoxin is ω-conotoxin GVIA.

11. The method of 4, wherein the antiepileptic drug is a botulinum toxin.

12. The method of claim 4, wherein the antiepileptic drug is an excitotoxin.

13. The method of claim 12, wherein the excitotoxin is ibotenate.

14. The method of claim 1, wherein the antiepileptic drug solution further comprises a tracer molecule.

15. The method of claim 1, wherein the antiepileptic drug solution further comprises liposomes suitable for CED.

16. The method of claim 1, wherein CED is administered through multiple distal infusion catheters with discharge ports situated in different regions of epileptic focus or in different foci, if more than one foci are in need of treatment.

17. The method of claim 1, wherein CED is performed using an apparatus comprising a pump and a catheter, wherein the pump is external to the body, wherein the catheter enters the body percutaneously, and wherein a proximal end of the catheter is coupled to the pump.

18. The method of claim 1, wherein CED is performed using an apparatus comprising a combination implantable pump and catheter, a control unit, and a sensor that detects electrical interictal or ictal seizure activity and which, through the control unit, commands the pump to deliver toxin solution when needed.

19. The method of claim 18, wherein the control unit stores a database of the sensor output signals and provides a signal to the pump based on the continuously updated database.
